(19) United States
(12) Patent Application Publication
(10) Pub. No.: US 2025/0228869 A1
(43) Pub. Date: Jul. 17, 2025

(54) Benzodiazepine Pyrazolo Carboxamides as N-protein Inhibitors

(71) Applicant: Pfizer Inc., New York, NY (US)

(72) Inventors: Jan Antoinette Cusi Romero Adams, Arlington, MA (US); Samit Kumar Bhattacharya, Waltham, MA (US); Matthew Jeremy Barrett, Darlington (GB); Alexandre Francois Bedernjak, Sedgefield (GB); George Stuart Cockerill, Richmond (GB); James Arthur Dudley Good, Barcelona (ES); Amit Kalgutkar, Waltham, MA (US); Raman Sharma, Groton, CT (US); Joseph Tillotson, Norwood, MA (US)

(73) Assignee: Pfizer Inc., New York, NY (US)

(21) Appl. No.: 19/023,946

(22) Filed: Jan. 16, 2025

Related U.S. Application Data
(60) Provisional application No. 63/621,331, filed on Jan. 16, 2024.

(51) Int. Cl. A61K 31/5513; A61K 31/4178; A61P 31/14

(52) U.S. Cl. CPC A61K 31/5513; A61K 31/4178; A61P 31/14

(57) ABSTRACT

The invention relates to benzodiazepine pyrazolo carboxamides and pharmaceutically acceptable salts thereof as defined in the description; to their use in medicine; to compositions containing them; to processes for their preparation; and to intermediates used in such processes. The compounds of invention inhibit the activity RSV N-proteins and may be useful in the treatment, prevention, suppression, and amelioration of viral infections such as RSV.

### FIELD OF THE INVENTION

The present invention relates to benzodiazepine pyrazolo carboxamides compounds and to their use in treating or preventing a respiratory syncytial virus (RSV) infection.

### BACKGROUND OF THE INVENTION

RSV is a negative-sense, single-stranded RNA virus of the Paramyxoviridae family. RSV is readily transmitted by secretions from an infected person via surfaces or hand-to-hand transfer. Unlike influenza, it is not transmitted by small-particle aerosols. Following successful inoculation, the incubation period is between four and six days during which time the virus spreads from the nasopharynx to the lower respiratory tract by fusion of infected with uninfected cells and by sloughing of the necrotic epithelium. In infants, coupled with increased mucus secretion and oedema, this can lead to mucus plugging causing hyper-inflation and collapse of distal lung tissue indicative of bronchiolitis. Hypoxia is common and the ability to feed is often impaired because of respiratory distress. In RSV pneumonia, inflammatory infiltration of the airways consists of mononuclear cells and is more generalized, with involvement of the bronchioles, bronchi and alveoli. The duration and degree of viral shedding has been found to correlate with the clinical signs and severity of disease.

RSV is the leading cause of serious respiratory tract infections in infants and young children throughout the world. The highest morbidity and mortality occur in those born prematurely and for those with chronic lung or heart disease, although many infants hospitalized for RSV infection are otherwise healthy. Severe RSV infection in infancy can lead to several years of recurrent wheezing and is linked to the later development of asthma.

RSV is also a major cause of morbidity and mortality in the elderly and in immunocompromised children and adults as well as those with chronic obstructive pulmonary disease (COPD) and congestive heart failure (CHF).

Current anti-RSV treatments include ribavirin, the use of which has concerns because of toxicity, teratogenicity potential and limited efficacy and palivizumab, a monoclonal antibody to RSV. Such use of palivizumab is a prophylactic, rather than therapeutic, treatment of RSV. Although this antibody is often effective, its use is restricted to preterm infants and infants at high risk. Indeed, its limited utility means that it is unavailable for many people in need of anti-RSV treatment. There is therefore an urgent need for effective alternatives to existing anti-RSV treatment.

Small molecules have also been proposed as inhibitors of RSV. These include benzimidazoles and benzodiazepines. For instance, benzimidazole inhibitors of RSV are disclosed in WO 02/062290 and WO 03/053344 (Squibb Bristol Myers Co); WO 2010/103306 (Astrazeneca UK Ltd); and WO 2013/068769, WO 2016/055780, WO 2019/016566 and WO 2019/122928 (ReViral Limited). The discovery and initial development of RSV604, a benzodiazepine compound having sub-micromolar anti-RSV activity, is described in Antimicrobial Agents and Chemotherapy, September 2007, 3346-3353 (Chapman et al). Benzodiazepine inhibitors of RSV are also disclosed in publications including WO 2004/026843 and WO 2005/089770 (Arrow Therapeutics Limited); WO 2016/166546 and WO 2018/033714 (Durham University); WO 2017/015449, WO 2018/129287 and WO 2018/226801 (Enanta Pharmaceuticals, Inc.); and WO 2021/079121, WO 2021/084280, WO 2021/032992, WO 2022/008911 and WO 2022/008912 (ReViral Limited).

RV299, a novel N-protein inhibitor in Phase 1 was recently discontinued and to date there has yet to be an approved N-protein inhibitor to treat RSV on the market.

Accordingly, there remains a need for improved treatments for RSV viral infections. The compounds and methods of the present invention have one or more advantages, including improved potency with an unexpectedly low predicted human dose, improved safety, and unexpectedly improved metabolic stability compared to other known N-protein inhibitors.

### SUMMARY OF THE INVENTION

The present invention provides, in part, the compounds of Formula (I) and pharmaceutically acceptable salts thereof. The compounds of Formula (I) inhibit the activity of viral N-proteins such as in RSV and may be useful in the treatment, prevention, suppression and amelioration of viral infections, including RSV. Also provided are pharmaceutical compositions and medicaments, comprising the compound or pharmaceutically acceptable salts of the invention, alone or in combination with additional RSV therapeutic agents. The present invention also provides, in part, methods for preparing the compound, pharmaceutically acceptable pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided Formula (I):

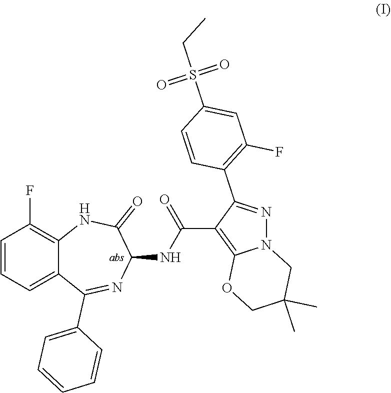 [SMILES: CCS(=O)(=O)c1ccc(-c2nn3c(c2C(=O)N[C@H]2N=C(c4ccccc4)c4cccc(F)c4NC2=O)OCC(C)(C)C3)c(F)c1]

wherein R¹ or R² are independently selected from the group consisting of —CH₃, —CD₃, and —CH₂OH; or a pharmaceutically acceptable salt thereof.

Described below are embodiments of the invention, where for convenience E1 is identical to the embodiment of Formula (1) or pharmaceutically acceptable salts thereof, provided above.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.
E1 is the compound of Formula (1) as defined above.
E2 is the compound of E1, or a pharmaceutically acceptable salt thereof, wherein R¹ is —CH₃ or —CD₃, and R² is —CH₃, CD₃ or —CH₂OH.
E3 is the compound of E1, or a pharmaceutically acceptable salt thereof, wherein R¹ is —CH₃ and R² is —CH₃ or —CH₂OH.
E4 is a compound or a pharmaceutically acceptable salt thereof, wherein R¹ is —CH₃ and R² is —CH₂OH.
E5 is the compound of claim 4 that is a diastereomer represented by

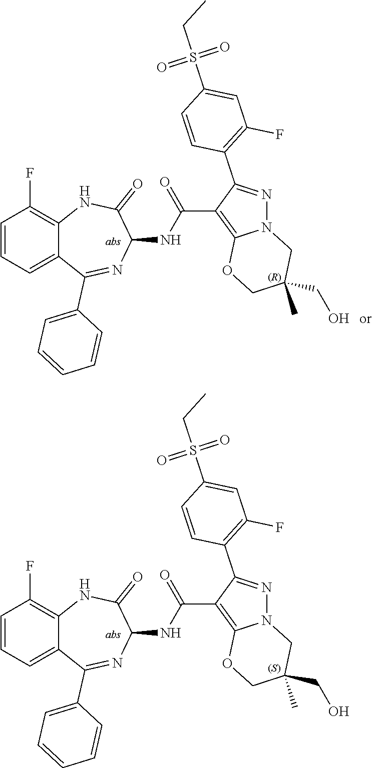

or pharmaceutically acceptable salt thereof.
E6 is the compound 2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide:

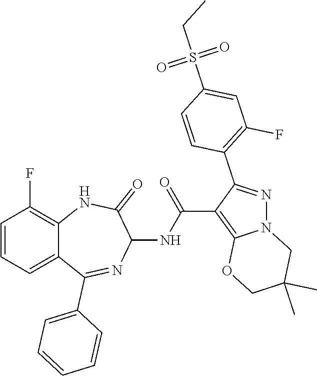 [SMILES: CCS(=O)(=O)c1ccc(-c2nn3c(c2C(=O)NC2N=C(c4ccccc4)c4cccc(F)c4NC2=O)OCC(C)(C)C3)c(F)c1]

or a pharmaceutically salt thereof.
E7 is the compound (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide:

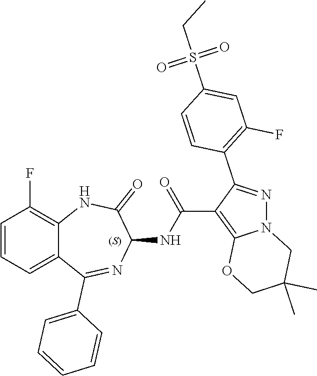

or a pharmaceutically acceptable salt thereof.
E8 is the compound (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide:

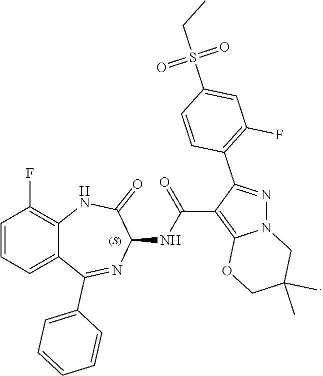

E9 is a pharmaceutically acceptable salt of (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide.
E10 is a pharmaceutical composition comprising the compound according to any one of embodiments E1 to E8 or a pharmaceutically acceptable salt thereof, and one or more pharmaceutically acceptable excipients.
E11 is a pharmaceutical composition comprising a pharmaceutically acceptable salt of any one of embodiments E1 to E9, and one or more pharmaceutically acceptable excipients.
E12 is a method for treating a RSV infection, the method comprising administering to a subject in need thereof a therapeutically effective amount of the compound or pharmaceutically acceptable salt thereof according to any one of embodiments E1 to E9.
E13 is the method of embodiment E12 further comprising administering a therapeutically effective amount of an additional RSV therapeutic agent.
E14 is the method of embodiment E13 wherein the additional RSV therapeutic agent is selected from the group consisting of sisunatovir, ziresovir, EDP-938, EDP-323, JNJ-64417184, PC786, S-337395, MRK-1, JNJ-8003, BI-D, AVG-158, AVG-233, AZ-27, molnupiravir, remdesivir, obeldesivir, and ribavirin.
E15 is the method of embodiment E14 wherein the additional RSV therapeutic agent is selected from the group consisting of sisunatovir, ziresovir, EDP-938, EDP-323, JNJ-64417184, PC786, S-337395, MRK-1, JNJ-8003, BI-D, AVG-158, AVG-233, and AZ-27.
E16 is the method of embodiment E15 wherein the additional RSV therapeutic agent is sisunatovir.
E17 is the method of any of embodiment E12 to E16 wherein the compound is (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide.
E18 is the compound or pharmaceutically acceptable salt thereof according to any one of embodiment E1 to E9 for use as a medicament.
E19 is the compound or pharmaceutically acceptable salt thereof according to any one of embodiment E1 to E9 for use in the treatment of a RSV infection.
E20 is the compound or pharmaceutically acceptable salt thereof according to any one of embodiment E1 to E9 for the manufacture of a medicament for the treatment of a RSV infection.

Each of the embodiments described above can be combined with any other embodiment described herein not inconsistent with the embodiment with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiments described herein.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

“Compounds of the invention” include the compounds of Formula (1) and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 100 mg) means that the parameter may vary by as much as 10% below or above the stated numerical value for that parameter. For example, a dose of about 100 mg means 100 mg±10%, i.e., it may vary between 90 mg and 110 mg.

The term “pharmaceutically acceptable” means the substance (e.g., the compound described herein) and any solvate or hydrate thereof, or composition containing the substance or solvate or hydrate of the invention is suitable for administration to a subject or patient.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula 1.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see PAULEKUHN, G. S., et al., “Trends in Active Pharmaceutical Ingredient Salt Selection Based on Analysis of the Orange Book Database,” Journal of Medicinal Chemistry, 2007, 50(26):6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃—Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

The compounds of the invention may exhibit polymorphism and/or one or more kinds of isomerism (e.g. optical, geometric or tautomeric isomerism). The compounds of the invention may also be isotopically labelled. Such variation is implicit to the compounds of the invention defined as they are by reference to their structural features and therefore within the scope of the invention.

### Tautomers

Compounds of the invention where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Stereoisomers

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). The resulting diastereomeric mixture may be separated by chromatography and/or fractional crystallization and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein). When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the invention can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to:
(i) where the compound of the invention contains an alkyl group or a hydroxyalkyl derivative thereof (—CH->—COH):

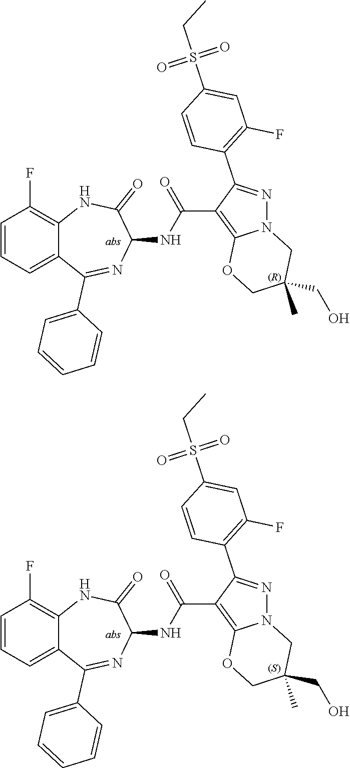

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

A “pharmaceutical composition” refers to a mixture of one or more of the compounds of the invention, or a pharmaceutically acceptable salt, solvate, hydrate or prodrug thereof as an active ingredient, and one or more pharmaceutically acceptable excipient.

The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, and liposomes. The form depends on the intended mode of administration and therapeutic application.

Typical compositions are in the form of injectable or infusible solutions, such as compositions similar to those used for passive immunization of humans with antibodies in general. One mode of administration is parenteral (e.g. intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dose form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral administration may be in a spray dried dispersion form. In another embodiment, the oral dose form is sub-lingual, such as, for example, a lozenge. In such solid dosage forms, the compounds of the invention are ordinarily combined with one or more adjuvants. Such capsules or tablets may contain a controlled release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the invention comprises a parenteral dose form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dose form. Such rectal dose form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or Igs; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250,500, 600, 750 or 1000 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient or from 50 to 500 milligrams. Intravenously, doses may range from about 0.01 to about 10 mg/kg/minute during a constant rate infusion.

Liposomes containing compounds of the invention may be prepared by methods known in the art, such as described in U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as those used in LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a compound of the invention with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and
(3) ameliorating the disease; for example, ameliorating a disease, condition or disorder in an individual that is experiencing or displaying the pathology or symptomatology of the disease, condition or disorder (i.e., reversing the pathology and/or symptomatology).

Typically, a compound of the invention is administered in an amount effective to treat a condition as described herein. The compounds of the invention can be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention and/or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Therapeutic Methods and Uses

The compounds are inhibitors of the RSV N-protein and are useful in the treatment, prevention, suppression and amelioration of RSV viral infections.

### Co-Administration

The compounds of the invention can be used alone, or in combination with one or more other RSV therapeutic agents. The invention provides any of the uses, methods or compositions as defined herein wherein the compound of the invention, or pharmaceutically acceptable salt thereof, is used in combination with one or more other known therapeutic agents to treat RSV. Such combinations may provide a greater clinical benefit than dosing any single alone. Examples of greater clinical benefits could include a larger reduction in RSV symptoms, a faster time to alleviation of symptoms, reduced lung pathology, a larger reduction in the amount of RSV in the patient (viral load), and decreased mortality.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration.

The phrases “concurrent administration,” “co-administration,” “simultaneous administration,” “sequential administration” and “administered simultaneously” mean that the compounds are administered in combination.

A compound of the invention and the one or more other RSV therapeutic agents may be administered as a fixed or non-fixed combination of the active ingredients. The term “fixed combination” means a compound of the invention, or a pharmaceutically acceptable salt thereof, and the one or more RSV therapeutic agents, are both administered to a subject simultaneously in a single composition or dosage. The term “non-fixed combination” means that a compound of the invention, or a pharmaceutically acceptable salt thereof, and the one or more RSV therapeutic agents are formulated as separate compositions or dosages such that they may be administered to a subject in need thereof simultaneously or sequentially with variable intervening time limits, wherein such administration provides effective levels of the two or more compounds in the body of the subject.

In one embodiment, the compounds of this invention are administered in combination with an additional RSV therapeutic agent useful in treatment of RSV infections including the pharmaceutically acceptable salts of the specifically named agents and the pharmaceutically acceptable solvates of said agents and salts. Additional therapeutic RSV agents include those which act as F-protein, N-protein, L-protein, and nucleoside inhibitors. Examples of additional therapeutic RSV agents include F-protein inhibitors sisunatovir and ziresovir (Ark Bio), N-protein inhibitor EDP-938 (Enanta); non-nucleoside RSV polymerase inhibitors EDP-323 (Enanta), JNJ-64417184 (Johnson & Johnson/Janssen), PC786 (Pulmocide), S-337395 (Shionogi), MRK-1 (Merck), JNJ-8003 (Johnson & Johnson/Janssen), BI-D (Boehringer Ingelheim), AVG-158, (Aviragen), AVG-233 (Aviragen), AZ-27 (Astra Zeneca); and nucleoside inhibitors molnupiravir, remdesivir, obeldesivir, and ribavirin.

### Synthetic Methods

Compounds of the present invention may be synthesized by synthetic routes that include processes analogous to those well-known in the chemical arts, particularly in light of the description contained herein. The starting materials are generally available from commercial sources such as Sigma-Aldrich, Inc. (St. Louis, Missouri) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der orqanischen Chemie, 4, Aufl. ed. Springer-Verlag, Berlin, including supplements (also available via the Beilstein online database)). The compounds used herein, are related to, or are derived from compounds in which there is a large scientific interest and commercial need, and accordingly many such compounds are commercially available or are reported in the literature or are easily prepared from other commonly available substances by methods which are reported in the literature.

For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide a variety of derivatives and/or reaction conditions. In addition, many of the compounds prepared by the methods described below can be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

### EXAMPLES

In order that this invention may be better understood, the following examples are set forth. These examples are for purposes of illustration only and are not to be construed as limiting the scope of the invention in any manner.

### Experimental Procedures

The following illustrate the synthesis of various compounds of the present invention. All starting materials in these Intermediates and Examples are either commercially available or can be prepared by methods known in the art or as described herein.

All reactions were carried out using continuous stirring under an atmosphere of nitrogen or argon gas unless otherwise noted. When appropriate, reaction apparatuses were dried under dynamic vacuum using a heat gun, and anhydrous solvents (Sure-Seal™ products from Sigma-Aldrich Sigma-Aldrich, Inc. (St. Louis, Missouri) or DriSolv™ products from EMD Chemicals, Gibbstown, NJ) were employed. In some cases, commercial solvents were passed through columns packed with 4A molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optimizer microwaves. Reaction progress was monitored using thin-layer chromatography (TLC), liquid chromatography-mass spectrometry (LCMS), high-performance liquid chromatography (HPLC), and/or gas chromatography-mass spectrometry (GCMS) analyses. TLC was performed on pre-coated silica gel plates with a fluorescence indicator (254 nm excitation wavelength) and visualized under UV light and/or with I₂, KMnO₄, CoCl₂, phosphomolybdic acid, and/or ceric ammonium molybdate stains. LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluent was analyzed using a Waters ZQ mass spectrometer scanning in both positive and negative ion modes from 100 to 1200 Da. Other similar instruments were also used. HPLC data were generally acquired on an Agilent 1100 Series instrument, using the columns indicated, acetonitrile/water gradients, and either trifluoroacetic acid or ammonium hydroxide modifiers. GCMS data were acquired using a Hewlett Packard 6890 oven with an HP 6890 injector, HP-1 column (12 m×0.2 mm×0.33 μm), and helium carrier gas. The sample was analyzed on an HP 5973 mass selective detector scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were performed by chiral supercritical fluid chromatography (SFC), generally using Waters, Berger or Thar instruments; columns such as Chiral Technologies AD-H, ChiralPAK-AD, -AS, —IC, Chiralcel-OD, or -OJ columns; and CO₂ mixtures with methanol, ethanol, 2-propanol, or acetonitrile, alone or modified using trifluoroacetic acid or propan-2-amine. UV detection was used to trigger fraction collection. For syntheses referencing procedures in other Examples or Methods, purifications may vary: in general, solvents and the solvent ratios used for eluents/gradients were chosen to provide appropriate R_{f}s or retention times.

Mass spectrometry data are reported from LCMS analyses. Mass spectrometry (MS) was performed via atmospheric pressure chemical ionization (APCI), electrospray Ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, d) referenced to the deuterated solvent residual peaks (CDCl₃, 7.26 ppm; CD₃OD, 3.31 ppm; CD₃CN, 1.94 ppm; (CD₃)₂SO, 2.50 ppm; D₂O, 4.79 ppm). The peak shapes are described as follows: s, singlet; d, doublet; t, triplet; q, quartet; quin, quintet; m, multiplet; br s, broad singlet; app, apparent. Fluorine nuclear magnetic spectroscopy (¹⁹F NMR) chemical shifts are given in parts per million and were recorded on 300, 400, 500, or 600 MHz Varian, Bruker, or Jeol spectrometers. Analytical SFC data were generally acquired on Agilent or Berger analytical instruments as described above. Optical rotation data were acquired on a PerkinElmer model 343 polarimeter using a 1 dm cell. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially and used without further purification, or were prepared using methods known in the literature.

The terms “concentrated”, “evaporated”, and “concentrated in vacuo” refer to the removal of solvent at reduced pressure on a rotary evaporator with a bath temperature less than 60° C. The abbreviation “min” and “h” stand for “minutes” and “hours” respectively. The term “TLC” refers to thin-layer chromatography, “room temperature or ambient temperature” means a temperature between 18 to 25° C., “GCMS” refers to gas chromatography-mass spectrometry, “LCMS” refers to liquid chromatography-mass spectrometry, “UPLC” refers to ultra-performance liquid chromatography and “HPLC” refers to high-performance liquid chromatography, “SFC” refers to supercritical fluid chromatography.

HPLC, UPLC, LCMS, GCMS, and SFC retention times were measured using the methods noted in the procedures.

The compounds and intermediates described below were named using the naming convention provided with ChemDraw (Version 20.1.1.125, PerkinElmer Informatics, Inc., Shelton, Connecticut, USA) The naming convention provided with ChemDraw 20.1.1.125 is well known by those skilled in the art and it is believed that the naming convention provided with ChemDraw 20.1.1.125 generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

### Example 1: 2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (1)

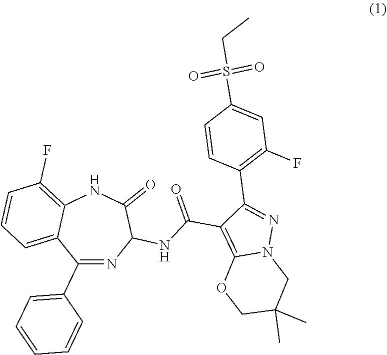 [SMILES: CCS(=O)(=O)c1ccc(-c2nn3c(c2C(=O)NC2N=C(c4ccccc4)c4cccc(F)c4NC2=O)OCC(C)(C)C3)c(F)c1]

### Intermediate 1: 3-Amino-9-fluoro-5-phenyl-1,3-dihydro-2H-benzo[e][1,4]diazepin-2-one (C1)

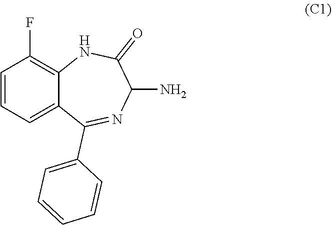 [SMILES: NC1N=C(c2ccccc2)c2cccc(F)c2NC1=O]

### Step 1: Synthesis of (2-Amino-3-fluorophenyl)(phenyl)methanone

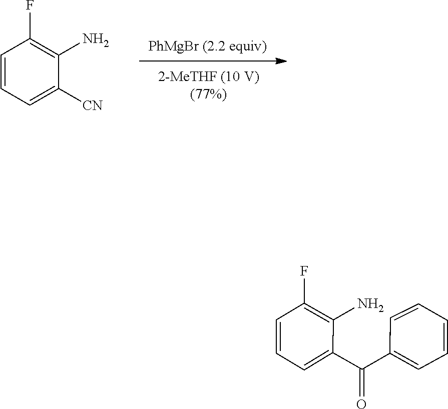 [SMILES: N#Cc1cccc(F)c1N ; Nc1c(F)cccc1C(=O)c1ccccc1]

2-MeTHF (1000 mL, 10 V) was charged into a reactor. 2-amino-3-fluorobenzonitrile (100 g, 735 mmol, 1.0 equiv) was added. The reactor was purged with nitrogen three times and the temperature was adjusted to between −5 and 5° C. (internal temperature was at 0° C.). A solution of PhMgBr in 2-MeTHF (2.8 M, 577 mL, 1616 mmol, 2.2 equiv) was added dropwise into the reactor between −5 and 5° C. over 30 min under nitrogen and exotherm was observed. The reactor was adjusted to between 2° and 30° C. (internal temperature at 25° C.), and the reaction mixture was stirred at this temperature for 16 hours (the reaction progress was monitored by HPLC of an aliquot quenched by 6 M HCl solution). The reactor was cooled down to between −5 and 5° C. and then 6 M HCl (735 mL, 6.0 equiv) was added dropwise (exotherm was observed). The reaction mixture was warmed to between 2° and 30° C. and stirred for another 16 hours. 6 M NaOH (300 mL) was added into the reaction slowly until pH reached between 8 and 9. The phases were separated and the aqueous layer was extracted with 2-MeTHF (5 V×2). The combined organic layers were washed with water (500 mL) and set aside to combine with another batch for further work-up and purification. This same reaction was performed again on a larger scale with 2-amino-3-fluorobenzonitrile (240 g, 735 mmol, 1 equiv), PhMgBr in 2-MeTHF (2.8 M, 2390 mL, 3880 mmol, 2.2 equiv) following the same procedures. The crude material after work-up in 2-MeTHF was combined with the previous batch and concentrated down keeping temperature below 40° C. and the solvent was switched to MTBE (5 V). Heptane (2.5 V) was added dropwise to the mixture and the solid was collected by filtration and the filter cake was washed with more heptane (0.5 V). The filter cake was dried between 35 to 45° C. for 16 hours to give the title compound (450.4 g, 77%) as a solid. LCMS calculated for C₁₃H₁₁FNO⁺ [M+H]⁺ 216.08, found 216.10.

### Step 2: Synthesis of 2-(1H-Benzo[d][1,2,3]triazol-1-yl)-2-(((benzyloxy)carbonyl)amino)acetic acid

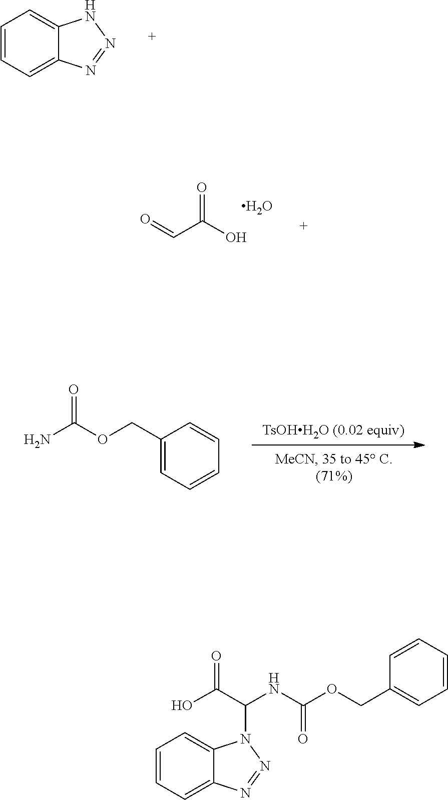

To a reactor was charged MeCN (500 mL, 5 V) and the temperature was adjusted between 15 to 25° C. Benzotriazole (100 g, 839 mmol, 1.0 equiv), glyoxylic acid monohydrate (77.3 g, 839 mmol, 1.0 equiv), benzyl carbamate (127 g, 839 mmol, 1.0 equiv) and finally p-toluenesulfonic acid monohydrate (TsOH·H2O, 2.89 g, 16.8 mmol, 0.02 equiv) were added with stirring. After the reaction mixture became a homogeneous solution, nitrogen gas was bubbled through for 5 minutes and the mixture was warmed to between 35 and 45° C. After stirring at this temperature for 17 hours, the reaction mixture was warmed to between 55 and 65° C. within 1 hour and stirred at this temperature for 0.5 hour. The temperature was allowed to slowly drop to between 35 and 45° C. again within 1 hour and stirred for 0.5 hour. The reaction mixture was further cooled to 20 and 30° C. and the solid was collected by filtration. After HPLC analysis of the solid and the filtrate, both components were recombined and concentrated to between 1 and 2V. The mixture was stirred in the previous reactor between 35 and 45° C. for 18 hours and cooled down to between 2° and 30° C. The precipitate was collected by filtration and further dried with heat to yield 196.6 g title product (72%, 99.1% liquid chromatography area percent, (LCAP) as a solid and used without further purification.

### Step 3: Synthesis of Benzyl (1-(1H-benzo[d][1,2,3]triazol-1-yl)-2-((2-benzoyl-6-fluorophenyl)amino)-2-oxoethyl)carbamate

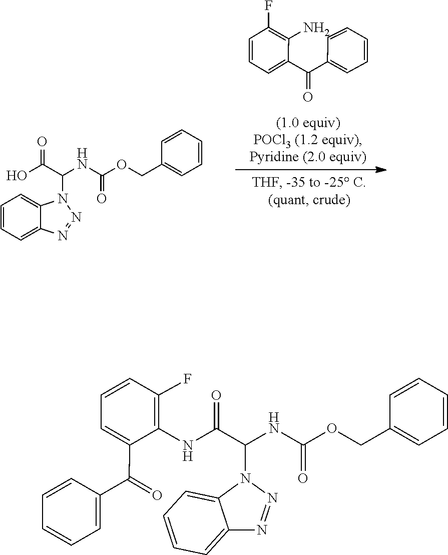 [SMILES: Nc1c(F)cccc1C(=O)c1ccccc1 ; O=C(NC(C(=O)O)n1nnc2ccccc21)OCc1ccccc1 ; O=C(NC(C(=O)Nc1c(F)cccc1C(=O)c1ccccc1)n1nnc2ccccc21)OCc1ccccc1]

2-(1H-Benzo[d][1,2,3]triazol-1-yl)-2-(((benzyloxy)carbonyl)amino)acetic acid (182 g, 558 mmol, 1.2 equiv) was charged into a reactor followed by THF (1000 L, 10 V). (2-Amino-3-fluorophenyl)(phenyl)methanone (100 g, 465 mmol, 1.0 equiv) was added and the reactor was purged with nitrogen gas three times. The reaction mixture was cooled down to between −35 and −25° C. and POCl₃ (51.0 mL, 85.5 g, 558 mmol, 1.2 equiv) was added. After stirring at the same temperature for 30 minutes, pyridine (74.8 mL, 73.5 g, 929 mmol, 2.0 equiv) was added dropwise over 2 hours. The reaction mixture was stirred between −35 and −25° C. for 16 hours. Water (2000 mL, 20 V) was charged into a second reactor followed by NaHCO₃ (390 g, 4650 mmol, 10.0 equiv). The reaction mixture from the first reactor was added to this NaHCO₃ solution while stirring, and temperature maintained between 2° and 30° C. The quenched mixture was stirred at the same temperature for 16 hours and EtOAc (1000 mL, 20 V) was added. The separated aqueous layer was extracted with EtOAc (500 mL, 10 V) and the combined organic layers were washed with water (10 V). The organic phase was concentrated to about 500 mL under vacuum below 40° C. and MeOH (5 V) was added. The mixture was further concentrated to 280 mL under vacuum below 40° C. and more MeOH (5 V) was added. The mixture was concentrated to dryness under vacuum below 40° C. to provide the title compound (243 g, quant, 93% LCAP) as a gum and used in the next step without purification.

### Step 4: Synthesis of Benzyl (9-fluoro-2-oxo-5-phenyl-2,3-dihydro-1H-benzo[e][1,4]diazepin-3-yl)carbamate

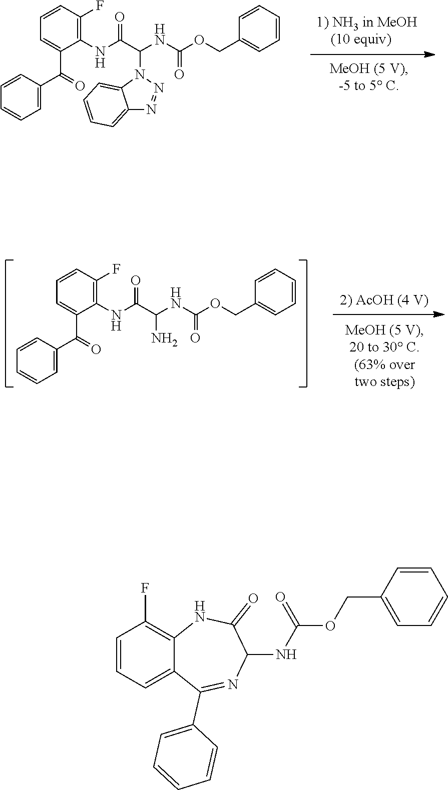

Benzyl (1-(1H-benzo[d][1,2,3]triazol-1-yl)-2-((2-benzoyl-6-fluorophenyl)amino)-2-oxoethyl)carbamate (crude 243 g, 464 mmol, 1.0 equiv) was charged into a reactor and MeOH (1215 mL, 5 V) was added. The reactor was purged with nitrogen gas three times and cooled down to between −5 and 5° C. NH₃ in MeOH (7 M, 663 mL, 4640 mmol, 10 equiv) was added. The reaction mixture was warmed to between 2° and 30° C. and stirred for 1 hour. The reaction mixture was concentrated under reduced pressure below 40° C. to provide the intermediate benzyl (1-amino-2-((2-benzoyl-6-fluorophenyl)amino)-2-oxoethyl)carbamate (240 g). MeOH (1200 mL, 5 V) was added and the mixture was concentrated to 2.5 V. Additional MeOH (600 mL, 2.5 V) was added. The temperature was re-adjusted to between 2° and 30° C. and AcOH (960 mL, 4 V) was charged. The reaction mixture was stirred between 2° and 30° C. for another 16 hours before water (240 mL, 1 V) was added. The solid filtered and rinsed with 5:1 MeOH:water (1 V). The filter cake was dried between 4° and 50° C. to provide the title compound (117 g, 63%, 99.6% LCAP) as a solid.

### Step 5: Synthesis of 3-Amino-9-fluoro-5-phenyl-1,3-dihydro-2H-benzo[e][1,4]diazepin-2-one (Cl)

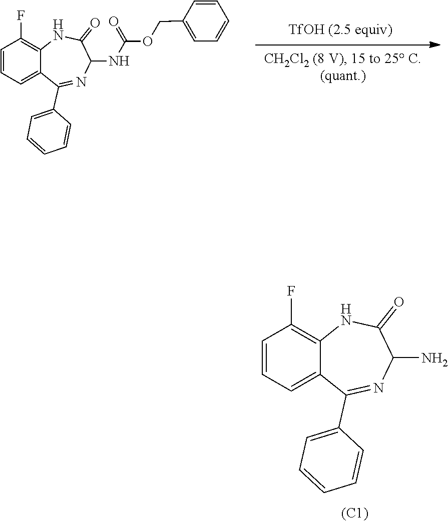

To a reactor was charged benzyl (9-fluoro-2-oxo-5-phenyl-2,3-dihydro-1H-benzo[e][1,4]diazepin-3-yl)carbamate (117 g, 290 mmol, 1.0 equiv), followed by CH₂Cl₂ (936 mL, 8V). The reactor was purged with nitrogen gas three times and the temperature was adjusted between 15 and 25° C. TfOH (58.0 mL, 109 g, 725 mmol, 2.5 equiv) was added dropwise and the reaction mixture was stirred between 15 and 25° C. for 16 hours. Water (234 mL) was added into the reactor while maintaining the same temperature. Meanwhile, a solution of NaHCO₃ was prepared by adding solid NaHCO₃ (73.1 g, 870 mmol, 3.0 equiv) into a second reactor with water (5 V). This NaHCO₃ solution was added dropwise into the first reactor with the reaction mixture and the pH was adjusted to between 7 and 8. The quenched reaction mixture was stirred between 15 and 25° C. for 2 hours. The resulting solid was filtered and washed with water (2 V) and EtOAc (0.5 V). The filter cake was dried between 35 and 45° C. for 16 hours to provide the title compound (79.8 g, quant, 100% LCAP but contaminated with trace NaOTf based on ¹⁹F NMR) as a solid. LCMS: calculated for C₁₅H₁₃FN₃O+[M+H]⁺ 270.10, found 270.1. ¹H NMR (400 MHz, (CD₃)₂SO) δ 7.61-7.48 (m, 4H), 7.48-7.37 (m, 2H), 7.25 (ddd, 1H), 7.10 (d, 1H), 4.32 (s, 1H). ¹⁹F NMR (376 MHz, (CD₃)₂SO) δ −124.16 (another small peak at −77.74 ppm indicating trace NaOTf).

The title compound C1 (CAS #1584714-99-7) may also be prepared using known procedures described in WO/2004/026843, WO/2005/090319, WO/2017/015449 and WO/2021/032992.

### Intermediate 2: Synthesis of 2-(4-(Ethylsulfonyl)-2-fluorophenyl)-6,6-dimethyl-6,7-dihydro-5H-pyrazolo[5,1-b][1,3]oxazine-3-carboxylic acid (C2)

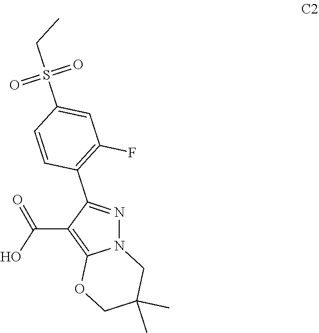 [SMILES: CCS(=O)(=O)c1ccc(-c2nn3c(c2C(=O)O)OCC(C)(C)C3)c(F)c1]

### Step 6: Synthesis of 5,5-dimethyl-1,3,2-dioxathiane 2,2-dioxide

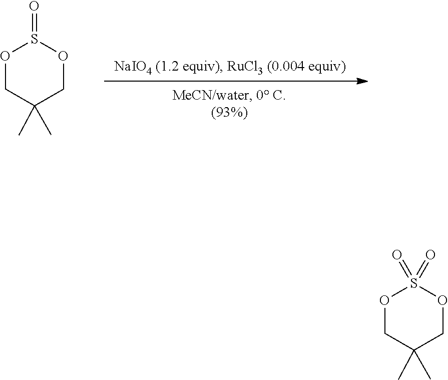

To a 3-necked round bottom flask equipped with overhead stirring and internal temperature monitoring was charged with 5,5-dimethyl-1,3,2-dioxathiane 2-oxide (8.0 g, 53.3 mmol, 1.0 equiv), acetonitrile (80 mL), and water (80 mL), and the mixture was cooled to an internal temperature of 0.4° C. while stirring. Ruthenium (Ill) chloride (0.32 mL, 48 mg, 0.21 mmol, 0.004 equiv) was charged before portion-wise addition of sodium metaperiodate (13.7 g, 63.9 mmol, 1.2 equiv) over 10 minutes while keeping internal temperature <14° C. The reaction mixture was stirred at an internal temperature of 12° C. for 15 minutes and then quenched with 300 mL of a saturated aqueous NaHSO₃ solution while keeping the internal temperature below 30° C. The reaction was diluted with EtOAc (500 mL) and the phases were separated. The organic layer was washed with saturated aq. NaHSO₃ (four times), brine (three times), dried over Na₂SO₄, concentrated in vacuo, and dried under high vacuum for four days to provide the title compound as a white solid (8.25 g, 93%). ¹H NMR (600 MHz, CDCl₃) δ 4.34 (s, 4H), 1.15 (s, 6H).

### Step 7: Synthesis of Ethyl 3,5-dibromo-1H-pyrazole-4-carboxylate

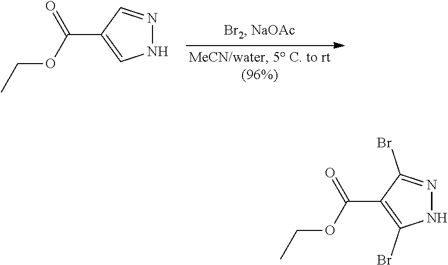

Sodium acetate trihydrate (127.0 g, 922 mmol, 6.8 equiv) was mixed with water (200 mL) and the solid was mostly dissolved. Ethyl 4-pyrazolecarboxylate (19.0 g, 135.58 mmol, 1.0 equiv) was dissolved in MeCN (100 mL). The sodium acetate mixture was charged to the MeCN solution of the ethyl 4-pyrazolecarboxylate. The resulting mixture was cooled to 0° C. and bromine (15.4 mL, 47.9 g, 298 mmol, 2.2 equiv) was added slowly. After 10 min, another portion of bromine (2.8 mL, 8.7 g, 54.2 mmol, 0.4 equiv) was added. The reaction mixture was warmed to rt and stirred for another 3 hours. Aqueous 10% sodium thiosulfate was added until the mixture turned light yellow. The mixture was diluted with EtOAc and 2-MeTHF and the layers were separated. The organic phase was concentrated in vacuo to give an off-white solid. This crude product was dried in the vacuum oven at 50° C. overnight. The resulting solid was suspended in water (150 mL) and stirred for 2 hours. After filtration, the solid was dried in vacuum oven at 50° C. for 4 hours and further dried until high vacuum at room temperature to remove residual AcOH. The title compound (38.72 g, 96%) was obtained as white solid. LCMS: calculated for C₆H₇Br⁷⁹Br⁷⁹N₂O₂⁺, C₆H₇Br⁷⁹Br⁸¹N₂O₂+ and C₆H₇Br⁸¹Br¹N₂O₂+[M+H]⁺ 296.89, 298.89 and 300.88, found 299.1, 297.1, 301, 1. ¹H NMR (400 MHz, CDCl₃) δ 4.37 (q, 2H), 1.40 (t, 3H).

### Step 8: Synthesis of Ethyl 3,5-dibromo-1-(3-hydroxy-2,2-dimethylpropyl)-1H-pyrazole-4-carboxylate

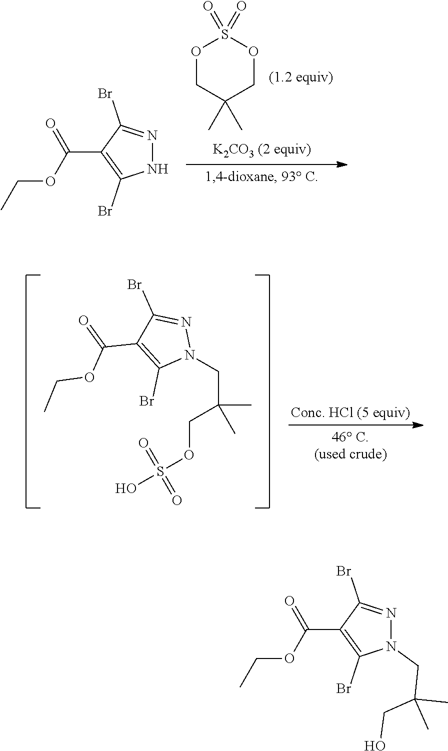

To a 3-necked 2 L round bottom flask equipped with a reflux condenser and overhead stirrer was added ethyl 3,5-dibromo-1H-pyrazole-4-carboxylate (50.0 g, 168 mmol, 1.0 equiv), 5,5-dimethyl-1,3,2-dioxathiane 2,2-dioxide (33.5 g, 201 mmol, 1.2 equiv), potassium carbonate (46.4 g, 336 mmol, 2.0 equiv), and 1,4-dioxane (500 mL, 10 V, 0.34 M). The heterogeneous white mixture was purged with 3 alternating cycles of vacuum/nitrogen gas and then stirred at an internal temperature of 93° C. for 21 hours. LCMS showed complete conversion to the desired product, and the reaction was then cooled to 0° C., and conc. HCl (12 M, 55 mL, 839 mmol, 4.0 equiv) was slowly added. The mixture was then stirred at 0° C. for 5 minutes, warmed to RT, and stirred for 1 hour. Additional conc. HCl (12 M, 14 mL, 1 equiv) was added and the mixture was stirred at 46° C. (internal temperature) for 2 hours. The reaction was cooled to 10° C. and slowly quenched with a saturated aqueous solution of NaHCO₃ (400 mL) until pH=9. The separated aqueous layer was extracted with EtOAc (500 mL×3). The combined organic layers were dried over MgSO₄, filtered and concentrated to provide the title compound (72.2 g, 84 wt % potency, containing small amounts of the reagent 5,5-dimethyl-1,3,2-dioxathiane 2,2-dioxide and EtOAc) as an orange oil, which was used in the next step without further purification. LCMS calculated for C₁₁H₁₇Br⁷⁹Br⁷⁹N₂O₃+, C₁₁H₁₇Br⁷⁹Br⁸¹N₂O₃+ and C₁₁H₁₇Br₈₁Br₈₁N₂O₃+[M+H]⁺ 382.96, 384.96 and 386.96, found 383.2, 385.2 and 387.2. ¹H NMR (400 MHz, CDCl₃) δ 4.35 (q, 2H), 4.12 (s, 2H), 3.30 (d, 2H), 2.91 (t, 1H), 1.39 (t, 3H), 0.95 (s, 6H).

### Step 9: Synthesis of Ethyl 2-bromo-6,6-dimethyl-6,7-dihydro-5H-pyrazolo[5,1-b][1,3]oxazine-3-carboxylate

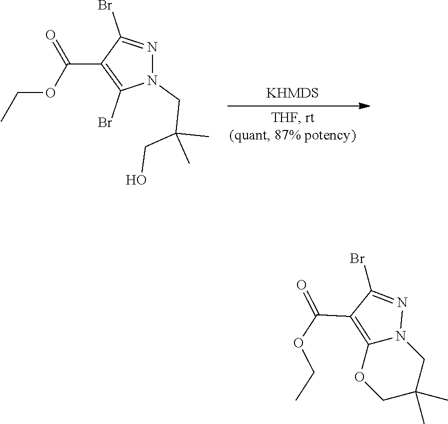 [SMILES: CCOC(=O)c1c(Br)nn(CC(C)(C)CO)c1Br ; CCOC(=O)c1c(Br)nn2c1OCC(C)(C)C2]

To a 3-necked 2 L round bottom flask equipped with an overhead stirrer was added ethyl 3,5-dibromo-1-(3-hydroxy-2,2-dimethylpropyl)-1H-pyrazole-4-carboxylate (72.0 g, 84 wt % potency, 160 mmol, 1.0 equiv) and THF (720 mL, 10 V, 0.22 M). The homogeneous yellow solution was purged with 3 alternating cycles of vacuum/nitrogen gas and then cooled to an internal temperature of 3° C. while stirring. KHMDS solution (1 M in THF, 161 mL, 160 mmol, 1.0 equiv) was added via addition funnel over 10 minutes, and the solution was then warmed to room temperature. The mixture was stirred for 2 hours before LCMS suggested complete conversion to the desired product. The reaction was then quenched by addition of water (300 mL), phases separated, and the aqueous layer was extracted with EtOAc (200 mL×3 mL). The combined organic layers were dried over MgSO₄, filtered, and filtrate concentrated to provide the title compound (40.0 g) as an orange oil, which was used in the next step without further purification (quant, 87 wt % potency). LCMS calculated for C₁₁H₁₆Br⁷⁹N₂O₃⁺ and C₁₁H₁₆Br⁸¹N₂O₃⁺ [M+H]⁺ calculated 303.03 and 305.03, found 303.3 and 305.3. ¹H NMR (400 MHz, CDCl₃) δ 4.31 (q, 2H), 4.03 (s, 2H), 3.79 (s, 2H), 1.36 (t, 3H), 1.15 (s, 6H).

### Step 10: Synthesis of 1-Bromo-4-(ethylsulfonyl)-2-fluorobenzene

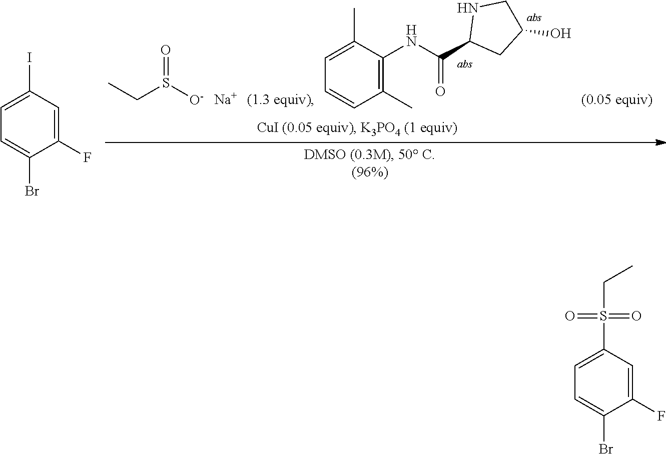

To a 3-necked round bottom flask equipped with a condenser, overhead stirrer, and internal temperature probe was added CuI (4.75 g, 24.9 mmol, 0.05 equiv), K₃PO₄ (106 g, 499 mmol, 1.0 equiv), sodium ethanesulfinate (75.2 g, 648 mmol, 1.3 equiv), and then a solution of 1-bromo-2-fluoro-4-iodobenzene (150 g, 498.5 mmol, 1.0 equiv) and (2S,4R)-N-(2,6-dimethylphenyl)-4-hydroxypyrrolidine-2-carboxamide (5.84 g, 24.9 mmol, 0.05 equiv) in DMSO (1.66 L, 0.3 M). The reaction was sparged with nitrogen gas for 20 minutes then heated at an internal temperature of 50° C. for 16 hours. The reaction was cooled to <10° C. (internal temperature) in an ice bath and then quenched with a 1:3 mixture of concentrated NH₄OH:saturated NH₄Cl (1 L) while keeping the internal temperature below 30° C. Solids precipitated during the quench. The solids were filtered off and washed with MTBE (1 L). The phases were separated and the aqueous layer was extracted MTBE (700 mL×3). The combined organic layers were collected and washed with saturated NH₄Cl (400 mL×2), brine (400 mL×2), dried over Na₂SO₄, and concentrated in vacuo to yield 1-bromo-4-(ethylsulfonyl)-2-fluorobenzene (128.4 g, 96%) as an off-white solid. This material was used as is in the following reaction without further purification. GCMS: calculated for C₈H₈⁷⁹BrFO₂S⁺ and C₈H₈³¹BrFO₂S⁺ [M]⁺ 265.94 and 267.94. found 265.9 and 267.9. ¹H NMR (400 MHz, (CD₃)₂SO) δ 8.05 (dd, 1H), 7.90 (dd, 1H), 7.67 (dd, 1H), 3.39 (q, 2H), 1.11 (t, 3H).

### Step 11: Synthesis of 2-(4-(Ethylsulfonyl)-2-fluorophenyl)-4,4,5,5-tetramethyl-1,3,2-dioxaborolane

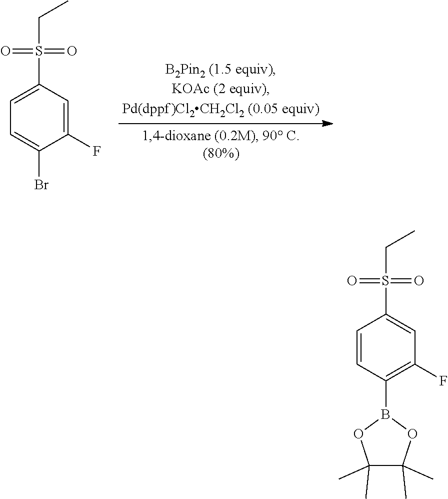 [SMILES: CCS(=O)(=O)c1ccc(Br)c(F)c1 ; CCS(=O)(=O)c1ccc(B2OC(C)(C)C(C)(C)O2)c(F)c1]

To a 3-necked round bottom flask equipped with a condenser, overhead stirrer, and internal temperature probe was added 1-bromo-4-(ethylsulfonyl)-2-fluorobenzene (128.4 g, 480 mmol, 1.0 equiv), 4,4,4′,4′,5,5,5′,5′-octamethyl-2,2′-bi(1,3,2-dioxaborolane) (183 g, 721 mmol, 1.5 equiv), KOAc (94.3 g, 961 mmol, 2.0 equiv), and 1,4-dioxane (1.2 L, 0.4 M). The reaction was sparged with nitrogen gas for 15 minutes before the addition of Pd(dppf)Cl₂·CH₂Cl₂ (13.4 g, 16.4 mmol, 0.025 equiv). The reaction was sparged with nitrogen gas for an additional 15 minutes and then heated at internal temperature of 90° C. for 16 hours. The reaction was cooled to room temperature, concentrated in vacuo, diluted with MTBE (1 L), filtered through a pad of celite then washed with 0.5 L MTBE (0.5 L). The combined organic layers were concentrated in vacuo. The residue was dissolved in EtOAc (900 mL) and treated with SiliaMetS® Thiol Metal Scavenger (60 g) then heated to reflux for 1.5 hours. The slurry was filtered. To the filtrate was added SiliaMetS® Thiol Metal Scavenger (60 g) and again heated to reflux for 1.5 hours. After filtration, the filtrate was treated with DARCO activated charcoal (55 g), which was removed by filtration through a Celite® pad. The resulting filtrate was concentrated in vacuo then diluted with heptane (1.1 L, 7.2 V). The slurry was heated to reflux for 2 hours to give a homogeneous solution. The solution was slowly cooled to room temperature, at which point solids precipitated from solution. The solids were filtered and dried under vacuum at 40° C. for 16 hours to provide 2-(4-(ethylsulfonyl)-2-fluorophenyl)-4,4,5,5-tetramethyl-1,3,2-dioxaborolane (126 g, 95.6% potency, adjusted yield 120.5 g, 80%) as an off-white solid. LCMS: calculated for [C₁₄H₂₁BFO₄S]⁺ [M+H]⁺ 315.2, but only observed for the corresponding to boronic acid calculated for [C₈H₁₁BFO₄S]⁺ [M+H]⁺ 233.04, found 233.2. ¹H NMR (600 MHz, (CD₃)₂SO) δ 7.91 (dd, 1H), 7.73 (dd, 1H), 7.67 (dd, 1H), 3.37 (q, 2H), 1.32 (s, 12H), 1.09 (t, 3H).

### Step 12: Synthesis of Ethyl 2-(4-(ethylsulfonyl)-2-fluorophenyl)-6,6-dimethyl-6,7-dihydro-5H-pyrazolo[5,1-b][1,3]oxazine-3-carboxylate

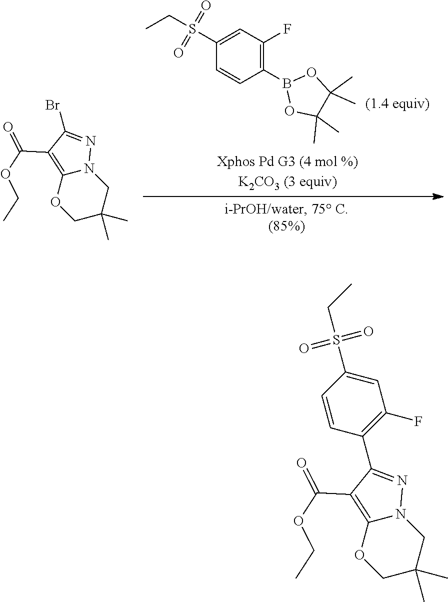 [SMILES: CCOC(=O)c1c(Br)nn2c1OCC(C)(C)C2 ; CCS(=O)(=O)c1ccc(B2OC(C)(C)C(C)(C)O2)c(F)c1 ; CCOC(=O)c1c(-c2ccc(S(=O)(=O)CC)cc2F)nn2c1OCC(C)(C)C2]

To a 3-necked round bottom flask equipped with a condenser, overhead stirring, and internal temperature monitoring was charged with a slurry of ethyl 2-bromo-6,6-dimethyl-6,7-dihydro-5H-pyrazolo[5,1-b][1,3]oxazine-3-carboxylate (30.0 g, 85.8 mmol, 1.0 equiv), 2-(4-(ethylsulfonyl)-2-fluorophenyl)-4,4,5,5-tetramethyl-1,3,2-dioxaborolane (39.6 g, 95% potency, 120 mmol, 1.4 equiv), XPhos Pd G3 (3.06 g, 3.43 mmol, 0.04 equiv) in i-PrOH (210 mL) followed by a slurry of K₂CO₃ (35.6 g, 257 mmol, 3.0 equiv) in water (42 mL), sequentially. The reaction was sparged with nitrogen gas for 20 minutes then stirred at internal temperature 75° C. for 1.5 hours. The reaction was cooled to room temperature and quenched with water (390 mL). The resulting solids were filtered and the filtrate was put aside to stir at room temperature overnight. Additional solids precipitated from the aqueous filtrate and the solid was collected by filtration. Both batches of solids were individually treated with EtOAc (1 L, 300 mL) and SiliCycle SiliaMetS® Thiol 40-63 μm (loading: 1.47 mmol/g) (15 g, 5 g) for 2.5 hours at reflux. The mixtures were filtered and concentrated in vacuo to provide yellow solids. The first batch was recrystallized in i-PrOH (350 mL) to provide the title compound (24.6 g, 70%) as a white solid. The second batch was recrystallized in i-PrOH (350 mL) followed by another recrystallization in i-PrOH (120 mL) to provide the title compound (5.37 g, 15%) as a white solid. The overall yield was 30.0 g (85%). LCMS: calculated for C₁₉H₂₄FN₂O₅S⁺ [M+H]⁺ 411.14, found 411.4. ¹H NMR (600 MHz, (CD₃)₂SO) δ 7.80-7.74 (m, 2H), 7.74-7.68 (m, 1H), 4.16 (s, 2H), 4.03 (q, 2H), 3.94 (s, 2H), 3.41 (q, 2H), 1.13 (t, 3H), 1.08 (s, 6H), 1.04 (t, 3H).

### Step 13: Synthesis of 2-(4-(Ethylsulfonyl)-2-fluorophenyl)-6,6-dimethyl-6,7-dihydro-5H-pyrazolo[5,1-b][1,3]oxazine-3-carboxylic acid (C2)

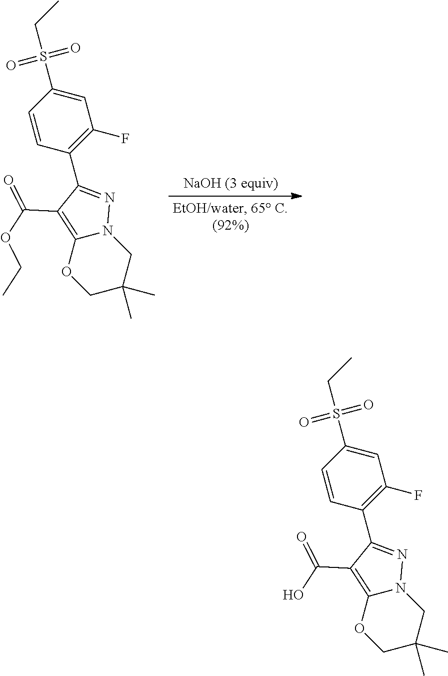 [SMILES: CCOC(=O)c1c(-c2ccc(S(=O)(=O)CC)cc2F)nn2c1OCC(C)(C)C2 ; CCS(=O)(=O)c1ccc(-c2nn3c(c2C(=O)O)OCC(C)(C)C3)c(F)c1]

To a 3-neck round bottom flask equipped with a condenser, overhead stirring and internal temperature monitoring was charged with ethyl 2-(4-(ethylsulfonyl)-2-fluorophenyl)-6,6-dimethyl-6,7-dihydro-5H-pyrazolo[5,1-b][1,3]oxazine-3-carboxylate (30.7 g, 74.8 mmol, 1.0 equiv), ethanol (219 mL), and aqueous NaOH (1 M, 220 mL, 220 mmol, 3 equiv) to give a white slurry. The mixture was stirred at internal temperature 65° C. for 18 hours. The solution was cooled to room temperature then acidified to pH 2 via dropwise addition of 12 M aqueous HCl. The resulting slurry was stirred at room temperature for 2 hours then filtered and washed water (300 mL×2). The solid was suspended in THF (200 mL) and refluxed for 4 hours. The slurry was cooled to room temperature and filtered, then dried in a vacuum oven at 45° C. to provide the title compound (26.3 g, 92%) as a white solid. LCMS: calculated for C₁₇H₂₀FN₂O₅S⁺ [M+H]⁺ 383.11, found 383.3. ¹H NMR (600 MHz, (CD₃)₂SO) δ 12.00 (s, 1H), 7.79-7.73 (m, 2H), 7.72-7.67 (m, 1H), 4.14 (s, 2H), 3.93 (s, 2H), 3.41 (q, 2H), 1.14 (t, 3H), 1.08 (s, 6H)

### Step 14: 2-(4-(Ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (1)

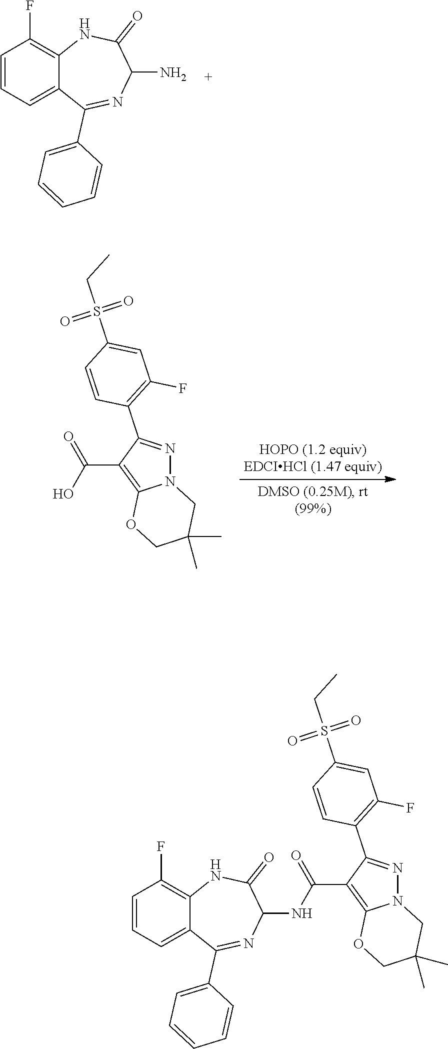 [SMILES: NC1N=C(c2ccccc2)c2cccc(F)c2NC1=O ; CCS(=O)(=O)c1ccc(-c2nn3c(c2C(=O)O)OCC(C)(C)C3)c(F)c1 ; CCS(=O)(=O)c1ccc(-c2nn3c(c2C(=O)NC2N=C(c4ccccc4)c4cccc(F)c4NC2=O)OCC(C)(C)C3)c(F)c1]

To an amber solution of 2-(4-(ethylsulfonyl)-2-fluorophenyl)-6,6-dimethyl-6,7-dihydro-5H-pyrazolo[5,1-b][1,3]oxazine-3-carboxylic acid (1500.0 mg, 92.9 wt % potency, 3.64 mmol, 1 equiv) in DMSO (15.0 mL, 11 V, 0.25 M) in a 100 mL round bottom flask was added 2-hydroxypyridine-N-oxide (485.8 mg, 4.37 mmol, 1.2 equiv). The reaction mixture formed a light red slightly cloudy solution. N-(3-dimethylaminopropyl)-N′-ethylcarbodiimide hydrochloride (EDCl·HCl, 1.027 g, 5.35 mmol, 1.47 mmol) was added and the resulting solution was stirred at room temperature for 50 minutes. LCMS indicated complete consumption of the carboxylic acid. 3-Amino-9-fluoro-5-phenyl-1,3-dihydro-2H-benzo[e][1,4]diazepin-2-one (1.255 g, 94.6 wt % potency, 4.41 mmol, 1.21 equiv) in five portions over 6 minutes. The reaction mixture was stirred at room temperature for 2 hours and during this time the mixture turned from a suspension into a clear solution. LCMS showed almost complete conversion to the desired product. The reaction was quenched by additional funnel of water (30 ml, 17 V) at 15° C. over 10 min and the resulting free flowing slurry was warmed to room temperature over 30 minutes. The precipitate was collected by filtration, rinsed with water (30 mL×5 for the reaction flask and 3×30 mL×3 for the filter cake). The filter cake was dried by vacuum under nitrogen gas overnight to provide the title compound (2410 mg, 99%) as a solid. LCMS calculated for C₃₂H₃₀F₂N₅O₅S⁺ [M+H]⁺ 634.19, found 634.5. ¹H NMR (600 MHz, (CD₃)₂SO) δ 10.95 (brs, 1H), 7.98 (d, 1H), 7.74-7.65 (m, 3H), 7.57 (ddd, 1H), 7.54-7.51 (m, 1H), 7.49-7.42 (m, 4H), 7.30 (ddd, 1H), 7.13 (d, 1H), 5.37 (d, 1H), 4.37 (s, 2H), 4.01 (s, 2H), 3.37 (q, 2H), 1.16 (s, 3H), 1.15 (s, 3H), 1.11 (t, 3H). ¹⁹F NMR (564 MHz, (CD₃)₂SO) δ −109.78, −123.35.

### Example 2 and 3: (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (2) and (R)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (3)

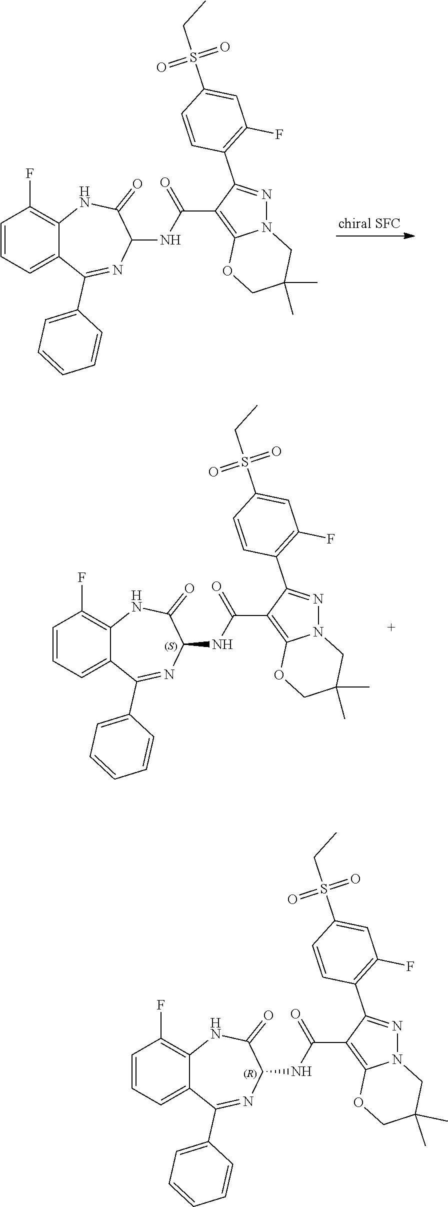

The 2.41 g racemic 2-(4-(Ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide was triturated in 6:1 water:MeCN (33 ml, 14V) at room temperature for 2 hours, filtered and dried at under high vacuum over the weekend. The resulting solid containing the two enantiomers of 2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (2.41 g, 3.80 mmol) was purified via preparative chiral SFC (instrument: Waters Prep 80, column: Regis Whelk-O (S,S) 250 mm×30.0 mm 5 u, mobile phase 60% CO₂/40% (1:1 MeCN:MeOH), flow rate: 150 mL/min, temperature: 40° C., backpressure: 100 bar). The chiral purity of the isomers was assessed using analytical chiral SFC (instrument: Agilent 1260 SFC-MS; column: Regis Whelk-O (S,S) 250 mm×4.6 mm 5 u; mobile phase: CO₂/(1:1 MeCN:MeOH), 95:5 to 0:100, flow rate 3.0 mL/min, backpressure: 120 Bar, detection at 210 nm). The title compound (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (retention time 4.63 min using the above analytical chiral SFC method, 1.160 g, 96%, 99.5% ee,) was isolated as a solid. Data for this (S)-enantiomer: LCMS calculated for C₃₂H₃₀F₂N₅O₅S⁺ [M+H]⁺ 634.19, found 634.5. ¹H NMR (600 MHz, (CD₃)₂SO) δ 10.96 (brs, 1H), 7.98 (d, 1H), 7.75-7.65 (m, 3H), 7.57 (ddd, 1H), 7.55-7.50 (m, 1H), 7.50-7.40 (m, 4H), 7.30 (ddd, 1H), 7.13 (dd, 1H), 5.37 (d, 1H), 4.37 (s, 2H), 4.01 (s, 2H), 3.37 (q, 2H), 1.16 (s, 3H), 1.15 (s, 3H), 1.11 (t, 3H). ¹⁹F NMR (564 MHz, (CD₃)₂SO) δ −109.77, −123.35. The other enantiomer (R)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (retention time 4.74 min using the above analytical chiral SFC method, 1.20 g, 99%, 98.4 ee %) was also isolated as a solid. Data for this (R)-enantiomer: LCMS calculated for C₃₂H₃₀F₂N₅O₅S⁺ [M+H]⁺ 634.19, found 634.5. ¹H NMR (600 MHz, (CD₃)₂SO) δ 10.96 (brs, 1H), 7.99 (d, 1H), 7.73-7.64 (m, 3H), 7.57 (dd, 1H), 7.54-7.50 (m, 1H), 7.50-7.41 (m, 4H), 7.29 (ddd, 1H), 7.13 (d, 1H), 5.37 (d, 1H), 4.37 (s, 2H), 4.01 (s, 2H), 3.37 (q, 2H), 1.16 (s, 3H), 1.15 (s, 3H), 1.11 (t, 3H). ¹⁹F NMR (564 MHz, (CD₃)₂SO) δ −109.76, −123.38.

### Example 4 and 5: (R)-2-(4-(ethylsulfonyl)-2-fluorophenyl)-N-((S)-9-fluoro-2-oxo-5-phenyl-2,3-dihydro-1H-benzo[e][1,4]diazepin-3-yl)-6-(hydroxymethyl)-6-methyl-6,7-dihydro-5H-pyrazolo[5,1-b][1,3]oxazine-3-carboxamide (4) and (S)-2-(4-(ethylsulfonyl)-2-fluorophenyl)-N-((S)-9-fluoro-2-oxo-5-phenyl-2,3-dihydro-1H-benzo[e][1,4]diazepin-3-yl)-6-(hydroxymethyl)-6-methyl-6,7-dihydro-5H-pyrazolo[5,1-b][1,3]oxazine-3-carboxamide (5)

### Step 1: Synthesis of Ethyl 3,5-dibromo-1-((2,2,5-trimethyl-1,3-dioxan-5-yl)methyl)-1H-pyrazole-4-carboxylate

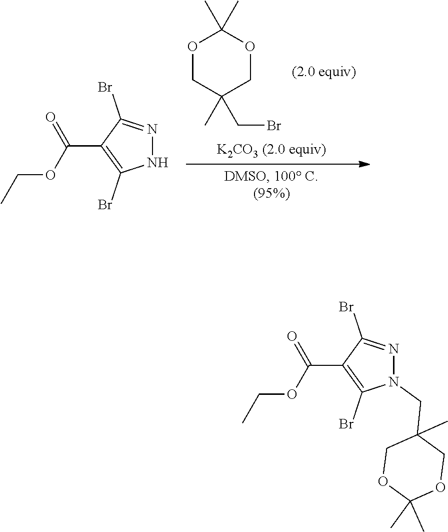 [SMILES: CCOC(=O)c1c(Br)n[nH]c1Br ; CC1(CBr)COC(C)(C)OC1 ; CCOC(=O)c1c(Br)nn(CC2(C)COC(C)(C)OC2)c1Br]

5-(bromomethyl)-2,2,5-trimethyl-1,3-dioxane can be prepared using literature procedure (ACS Appl. Mater. Interfaces 2023, 15, 2246-2255). To a vial was added ethyl 3,5-dibromo-1H-pyrazole-4-carboxylate (1.000 g, 3.356 mmol, 1.0 equiv), 5-(bromomethyl)-2,2,5-trimethyl-1,3-dioxane (1.498 g, 6.713 mmol, 2.0 equiv), K₂CO₃ (928 mg, 6.713 mmol, 2.0 equiv), and DMSO (5 mL). The reaction mixture was heated at 100° C. for 8 hours. The reaction was cooled to room temperature and then diluted with EtOAc and water. The organic layer was concentrated in vacuo and the residue was dissolved in DCM. Flash chromatography (0-25% EtOAc in heptane) provided ethyl 3,5-dibromo-1-((2,2,5-trimethyl-1,3-dioxan-5-yl)methyl)-1H-pyrazole-4-carboxylate (1.404 g, 95%) as a white waxy solid. ¹H NMR (400 MHz, (CD₃)₂SO) δ 4.34 (s, 2H), 4.26 (q, 2H), 3.63-3.51 (m, 4H), 1.37 (s, 3H), 1.35 (s, 3H), 1.30 (t, 3H), 0.79 (s, 3H).

### Step 2: Ethyl 3,5-dibromo-1-(3-hydroxy-2-(hydroxymethyl)-2-methylpropyl)-1H-pyrazole-4-carboxylate

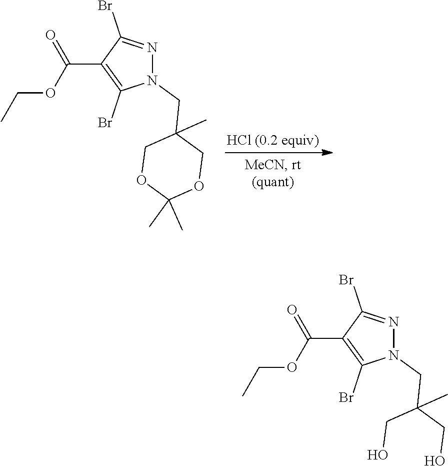 [SMILES: CCOC(=O)c1c(Br)nn(CC2(C)COC(C)(C)OC2)c1Br ; CCOC(=O)c1c(Br)nn(CC(C)(CO)CO)c1Br]

To a flask was added ethyl 3,5-dibromo-1-((2,2,5-trimethyl-1,3-dioxan-5-yl)methyl)-1H-pyrazole-4-carboxylate (1.40 g, 3.181 mmol, 1.0 equiv), MeCN (5 mL), and water (0.5 mL). HCl (12.1 M, 53 μL, 0.64 mmol, 0.2 equiv) was added to the reaction mixture. After 1 hour at room temperature, the reaction mixture was concentrated in vacuo and then the residue was dissolved in EtOAc. The organic layer was washed with saturated aqueous NaHCO₃, dried (MgSO₄), and then concentrated in vacuo to provide ethyl 3,5-dibromo-1-(3-hydroxy-2-(hydroxymethyl)-2-methylpropyl)-1H-pyrazole-4-carboxylate (1.273 g, 100%) as a yellow oil. LCMS: calculated for C₁₁H₁₇Br⁷⁹Br⁷⁹N₂O₄⁺, C₁₁H₁₇Br⁷⁹Br⁸¹N₂O₄⁺ and C₁₁H₁₇Br⁸¹Br⁸¹N₂O₄⁺ [M+H]⁺ 398.96, 400.95 and 402.95, found 399.1, 401.1 and 403.0. ¹H NMR (600 MHz, (CD₃)₂SO) δ 4.62 (s, 2H), 4.26 (q, 2H), 4.18 (s, 2H), 3.33 (d, 2H), 3.28 (d, 2H), 1.30 (t, 3H), 0.69 (s, 3H).

### Step 3: Ethyl 2-bromo-6-(hydroxymethyl)-6-methyl-6,7-dihydro-5H-pyrazolo[5,1-b][1,3]oxazine-3-carboxylate

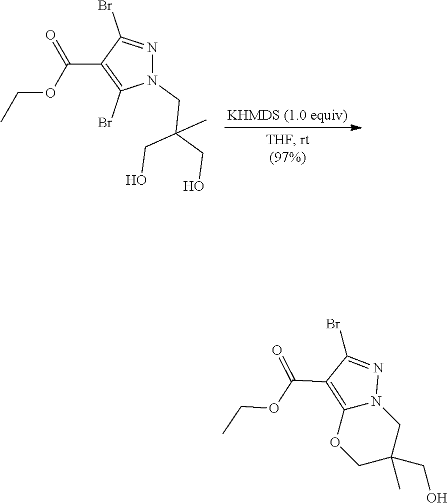 [SMILES: CCOC(=O)c1c(Br)nn(CC(C)(CO)CO)c1Br ; CCOC(=O)c1c(Br)nn2c1OCC(C)(CO)C2]

To a flask was added ethyl 3,5-dibromo-1-(3-hydroxy-2-(hydroxymethyl)-2-methylpropyl)-1H-pyrazole-4-carboxylate (1.10 g, 2.75 mmol, 1.0 equiv) and THF (10 mL). KHMDS (1 M in THF, 2.75 mL, 2.75 mmol, 1.0 equiv) was added to the reaction mixture slowly. After 20 min, the reaction mixture was quenched with water, concentrated in vacuo, and then the residue was dissolved in CH₂Cl₂. Flash chromatography (50-100% EtOAc in heptane) provided ethyl 2-bromo-6-(hydroxymethyl)-6-methyl-6,7-dihydro-5H-pyrazolo[5,1-b][1,3]oxazine-3-carboxylate (940 mg, contains 9.5 wt % EtOAc, calculated yield to be 851 mg, 97%) as a clear oil/white foam mixture. LCMS: calculated for C₁₁H₁₆Br⁷⁹N₂O₄⁺ and C₁₁H₁₆Br⁸¹N₂O₄₊[M+H]⁺ 319.03 and 321.0, found 319.2 and 321.1. ¹H NMR (600 MHz, (CD₃)₂SO) δ 5.08 (t, 1H), 4.26 (d, 1H), 4.17 (ddd, 2H), 4.14 (d, 1H), 3.97 (d, 1H), 3.76 (d, 1H), 3.32 (d, 2H), 1.23 (t, 3H), 0.98 (s, 3H).

### Step 4: Ethyl 2-(4-(ethylsulfonyl)-2-fluorophenyl)-6-(hydroxymethyl)-6-methyl-6,7-dihydro-5H-pyrazolo[5,1-b][1,3]oxazine-3-carboxylate

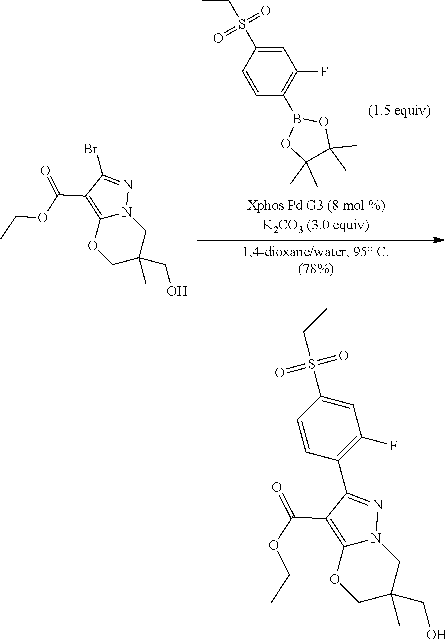 [SMILES: CCOC(=O)c1c(Br)nn2c1OCC(C)(CO)C2 ; CCS(=O)(=O)c1ccc(B2OC(C)(C)C(C)(C)O2)c(F)c1 ; CCOC(=O)c1c(-c2ccc(S(=O)(=O)CC)cc2F)nn2c1OCC(C)(CO)C2]

To a vial was added ethyl 2-bromo-6-(hydroxymethyl)-6-methyl-6,7-dihydro-5H-pyrazolo[5,1-b][1,3]oxazine-3-carboxylate (800 mg, 2.51 mmol, 1.0 equiv), 2-(4-(ethylsulfonyl)-2-fluorophenyl)-4,4,5,5-tetramethyl-1,3,2-dioxaborolane (1.02 g, 3.26 mmol, 1.5 equiv), K₂CO₃(1.04 g, 7.52 mmol, 3.0 equiv), and XPhos Pd G3 (170 mg, 0.201 mmol). The vial was purged with nitrogen gas, and then a nitrogen gas sparged mixture of 1,4-dioxane (10 mL) and water (2 mL) was added to the vial. The reaction mixture was heated at 95° C. for 2 hours, and then cooled to room temperature. The reaction mixture was extracted with EtOAc, the organic layer was concentrated in vacuo, and then the residue was dissolved in CH₂Cl₂. Flash chromatography (40-100% EtOAc in heptane) provided ethyl 2-(4-(ethylsulfonyl)-2-fluorophenyl)-6-(hydroxymethyl)-6-methyl-6,7-dihydro-5H-pyrazolo[5,1-b][1, 3]oxazine-3-carboxylate (836 mg, 78%) as a tan foamy solid. LCMS: calculated for C₁₉H₂₄FN₂O₆S⁺ [M+H]⁺ 427.13, found 427.3. ¹H NMR (600 MHz, (CD₃)₂SO) δ 7.81-7.74 (m, 2H), 7.74-7.70 (m, 1H), 5.11 (t, 1H), 4.31 (d, 1H), 4.19 (d, 1H), 4.08 (d, 1H), 4.03 (q, 2H), 3.88 (d, 1H), 3.41 (q, 2H), 3.37 (d, 2H), 1.13 (t, 3H), 1.04 (t, 3H), 1.03 (s, 3H).

### Step 5: Synthesis of Ethyl (S)-2-(4-(ethylsulfonyl)-2-fluorophenyl)-6-(hydroxymethyl)-6-methyl-6,7-dihydro-5H-pyrazolo[5,1-b][1,3]oxazine-3-carboxylate and ethyl (R)-2-(4-(ethylsulfonyl)-2-fluorophenyl)-6-(hydroxymethyl)-6-methyl-6,7-dihydro-5H-pyrazolo[5,1-b][1,3]oxazine-3-carboxylate

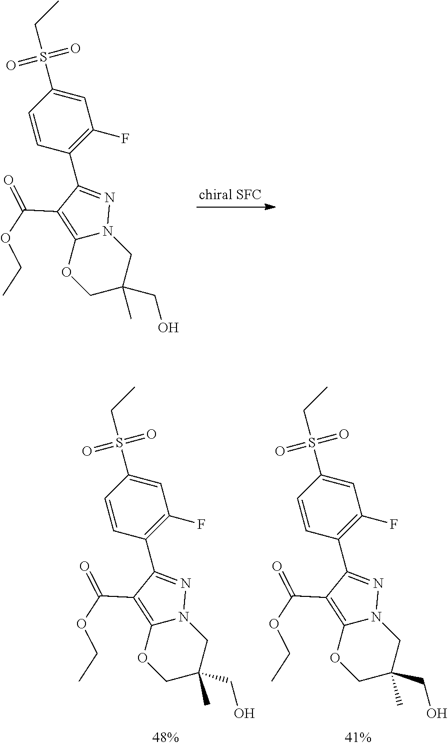

The two enantiomers of ethyl 2-(4-(ethylsulfonyl)-2-fluorophenyl)-6-(hydroxymethyl)-6-methyl-6,7-dihydro-5H-pyrazolo[5,1-b][1,3]oxazine-3-carboxylate were separated via preparative chiral SFC (instrument: Waters Prep 80, column: Chiral Technologies OX-H 250 mm×30.0 mm 5 u, mobile phase 80% CO₂/20% (2-propanol+0.2% 7N NH₃ in MeOH), flow rate: 100 mL/min, temperature: 50° C., backpressure: 100 bar). The chiral purity of the isomers was assessed using analytical chiral SFC (instrument: Agilent 1260 SFC-MS; column: Chiral Technologies OX-H 250 mm×4.6 mm 5 u; mobile phase: CO₂/(2-propanol+0.2% 7N NH₃ in MeOH), 95:5 to 40:60, flow rate 3.00 mL/min, backpressure: 100 bar, detection at 210 nm). Each isomer of ethyl 2-(4-(ethylsulfonyl)-2-fluorophenyl)-6-(hydroxymethyl)-6-methyl-6,7-dihydro-5H-pyrazolo[5,1-b][1,3]oxazine-3-carboxylate [peak 1 (Enantiomer 1): (400 mg, 48%, 100% ee, retention time 4.85 min), peak 2 (Enantiomer 2): (344 mg, 41%, 98.2% ee, retention time 5.01 min]was isolated as solids. The absolute stereochemistry of Enantiomer 1 and Enantiomer 2 were not assigned. Data for Enantiomer 1: retention time 4.85 min using above analytical chiral SFC method; ¹H NMR (600 MHz, (CD₃)₂SO) δ 7.85-7.74 (m, 2H), 7.74-7.69 (m, 1H), 5.11 (t, 1H), 4.31 (d, 1H), 4.19 (d, 1H), 4.08 (d, 1H), 4.03 (q, 2H), 3.88 (d, 1H), 3.41 (q, 2H), 3.37 (d, 2H), 1.13 (t, 3H), 1.04 (t, 3H), 1.03 (s, 3H). Data for Enantiomer 2: retention time 5.01 min using above analytical chiral SFC method; ¹H NMR (600 MHz, (CD₃)₂SO) δ 7.82-7.75 (m, 2H), 7.75-7.67 (m, 1H), 5.12 (t, 1H), 4.32 (d, 1H), 4.20 (d, 1H), 4.09 (d, 1H), 4.04 (q, 2H), 3.89 (d, 1H), 3.42 (q, 2H), 3.38 (d, 2H), 1.14 (t, 3H), 1.05 (t, 3H), 1.04 (s, 3H).

### Step 6: Synthesis of (R)-2-(4-(ethylsulfonyl)-2-fluorophenyl)-N-((S)-9-fluoro-2-oxo-5-phenyl-2,3-dihydro-1H-benzo[e][1,4]diazepin-3-yl)-6-(hydroxymethyl)-6-methyl-6,7-dihydro-5H-pyrazolo[5,1-b][1,3]oxazine-3-carboxamide and (S)-2-(4-(ethylsulfonyl)-2-fluorophenyl)-N-((S)-9-fluoro-2-oxo-5-phenyl-2,3-dihydro-1H-benzo[e][1,4]diazepin-3-yl)-6-(hydroxymethyl)-6-methyl-6,7-dihydro-5H-pyrazolo[5,1-b][1,3]oxazine-3-carboxamide

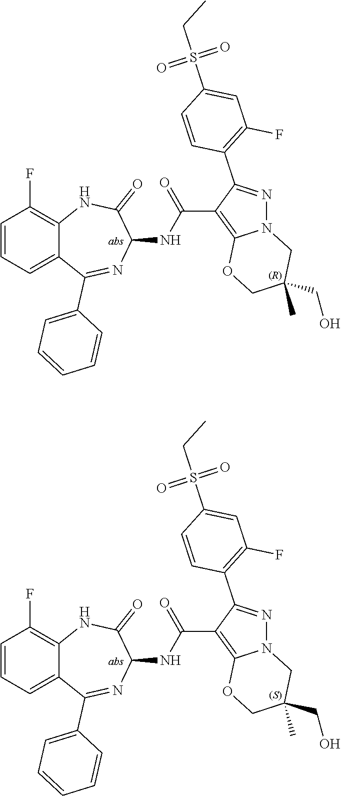

Starting with the separated enantiomers of ethyl 2-(4-(ethylsulfonyl)-2-fluorophenyl)-6-(hydroxymethyl)-6-methyl-6,7-dihydro-5H-pyrazolo[5,1-b][1,3]oxazine-3-carboxylate, the two title compounds were prepared in an analogous route as (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide and (R)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide (Examples 2 and 3). The absolute stereochemistry on the right hand side of the molecules were arbitrarily assigned. The absolute stereochemistry of the 9-fluorobenzodiazapine was assigned based on comparison with genuine samples of the metabolites generated from hepatocyte incubation of parent Example 2. Data for first metabolite Example 4: 100% ee; LCMS: calculated for C₃₂H₃₀F₂N₅O₆S⁺ [M+H]⁺ 650.19, found 650.5. ¹H NMR (600 MHz, (CD₃)₂SO) δ 7.97 (d, 1H), 7.76-7.64 (m, 3H), 7.57 (t, 1H), 7.52 (ddd, 1H), 7.50-7.42 (m, 4H), 7.32-7.26 (m, 1H), 7.13 (d, 1H), 5.37 (d, 1H), 5.18 (t, 1H), 4.52 (d, 1H), 4.39 (d, 1H), 4.15 (d, 1H), 3.95 (d, 1H), 3.45 (d, 2H), 3.37 (q, 2H), 1.11 (t, 3H), 1.09 (s, 3H). ¹⁹F NMR (564 MHz, (CD₃)₂SO) δ −109.75, −123.37.

Data for second metabolite Example 5: 100% ee; LCMS: calculated for C₃₂H₃₀F₂N₅O₆S⁺ [M+H]⁺ 650.19, found 650.5. ¹H NMR (600 MHz, (CD₃)₂SO) δ 10.95 (brs, 1H), 7.95 (d, 1H), 7.77-7.66 (m, 3H), 7.57 (ddd, 1H), 7.53 (ddd, 1H), 7.50-7.39 (m, 4H), 7.30 (ddd, 1H), 7.13 (d, 1H), 5.37 (d, 1H), 5.17 (t, 1H), 4.51 (d, 1H), 4.40 (d, 1H), 4.15 (d, 1H), 3.94 (d, 1H), 3.45 (d, 2H), 3.37 (q, 2H), 1.11 (t, 3H), 1.10 (s, 3H). ¹⁹F NMR (564 MHz, (CD₃)₂SO) δ −109.75, −123.36.

### Prophetic Deuterated Analogs (PDA) of Example 2

The compounds provided in Table 1 are prophetic deuterated analogs (PDA) of Example 2. The Formula (A) is the generic formula of deuterated Example 2, wherein Y¹, Y^{2a}, Y^{2b}, Y^{3a}, Y^{3b}, Y⁴, Y^{5a}, Y^{5b}, Y^{5c}, Y^{6a}, Y^{6b}, Y^{6c}, Y^{7a}, Y^{7b}, Y^{7c}, Y⁸, Y⁹, and Y¹⁰ are each independently H or D. The deuterated analogs of Example 2 in Table 1 are predicted based on the metabolic profile of Example 1, with MetaSite (moldiscovery.com/software/metasite/). Y¹, Y^{2a}, Y^{2b}, Y^{3a}, Y^{3b}, Y⁴, Y^{5a}, Y^{5b}, Y^{5c}, Y^{6a}, Y^{6b}, Y^{6c}, Y^{7a}, Y^{7b}, Y^{7c}, Y⁸, Y⁹, and Y¹⁰ are most likely to be metabolized position based on MetaSite predictions.

TABLE 1

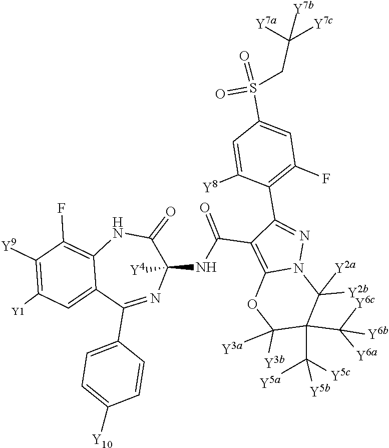

(A)

| PDA # | Y¹ | Y^{2a}-Y^{2b} | Y^{3a}-Y^{3b} | Y⁴ | Y^{5a}-Y^{5c} | Y^{6a}-Y^{6c} | Y^{7a}-Y^{7c} | Y⁸ | Y⁹ | Y¹⁰ |
|---|---|---|---|---|---|---|---|---|---|---|
| A-1 | D | H | H | H | H | H | H | H | H | H |
| A-2 | H | D | H | H | H | H | H | H | H | H |
| A-3 | H | H | D | H | H | H | H | H | H | H |
| A-4 | H | H | H | D | H | H | H | H | H | H |
| A-5 | H | H | H | H | D | H | H | H | H | H |
| A-6 | H | H | H | H | H | D | H | H | H | H |
| A-7 | H | H | H | H | H | H | D | H | H | H |
| A-8 | H | H | H | H | H | H | H | D | H | H |
| A-9 | H | H | H | H | H | H | H | H | D | H |
| A-10 | H | H | H | H | H | H | H | H | H | D |
| A-11 | D | D | H | H | H | H | H | H | H | H |
| A-12 | D | H | D | H | H | H | H | H | H | H |
| A-13 | D | H | H | D | H | H | H | H | H | H |
| A-14 | D | H | H | H | D | H | H | H | H | H |
| A-15 | H | D | D | H | H | H | H | H | H | H |
| A-16 | H | D | H | D | H | H | H | H | H | H |
| A-17 | H | D | H | H | D | H | H | H | H | H |
| A-18 | H | H | D | D | H | H | H | H | H | H |
| A-19 | H | H | D | H | D | H | H | H | H | H |
| A-20 | H | H | H | D | D | H | H | H | H | H |

Examples A-1 to A-20 in Table 1 may afford certain therapeutic advantages resulting from greater metabolic stability, for example increased in vivo half-life, reduced dosage requirements, reduced CYP450 inhibition (competitive or time dependent), or an improvement in therapeutic index or tolerability.

A person with ordinary skill may make additional deuterated analogs of Example 2 with different combinations of Y¹-Y¹⁰ as provided in Table 1. Such additional deuterated analogs may provide similar therapeutic advantages that may be achieved by the deuterated analogs.

### Determination of Distribution Coefficient (log D)

Shake flask method of determining log D—Miniaturized version of log D assay described in Stopher, D. and McClean, S. J. Pharm. Pharmacol. 1990, 42, 144.

Nadolol, mexiletine, propranolol, quinidine, amitriptyline, and chlorproazine were dissolved in DMSO to a concentration of 10 mM and used as controls in each plate. D-neo-Inositol, 5-[[(2E)-3-[4-[[(5E)-5-[[(3-chloro-2,6-difluorophenyl)methoxy]imino]-5,6-dideoxy-β-D-arabino-hexofuranosyl]oxy]-3-hydroxyphenyl]-2-methyl-1-oxo-2-propenyl]amino]-5-deoxy-1,2-O-methylene-(9Cl) (CAS 247598-17) is used as an Internal Standard (IS).

All liquid handling steps were performed by a Biomek FX. Buffer-saturated 1-octanol (149 μL/well) was added to duplicate 1 mL capacity 96-well plates. Test compounds (10 mM in DMSO; 2 μL/well) and controls (10 mM in DMSO; 2 μL/well) were transferred from Matrix 2D barcoded storage tubes to the 96-well plates.

Following the addition of 1-octanol saturated phosphate buffer (149 μL/well), the plates were sealed with silicone well cap-mats. The plates were vigorously mixed on their sides for 1 hour at a speed of 10 at room temperature on a plate shaker or 15 minutes on floor model at 2500 rpm then subjected to centrifugation at 2500 rpm (1006×g) for 15 minutes. After the cap-mats were removed from the plates, aliquots (4 μL/well) from the 1-octanol phase were transferred into new 1 mL capacity plates containing 1-octanol IS solution (796 μL/well). Aliquots (10 μL/well) from the buffer phase were transferred into other 1 mL capacity plates containing buffer IS solution (190 μL/well).

### LC-MS Conditions.

High-Throughput Micro-Flow Gradient LC-MS/MS (μfLC-MS/MS)

Samples were analyzed by micro-flow reversed-phase liquid chromatography coupled to tandem mass spectrometry (μfLC-MS/MS). An LS-1 sample delivery system was controlled by LeadScape software (Sound Analytics, Niantic CT) and interfaced with a SCIEX 6500/6500+ triple-quadrupole/Qtrap mass spectrometer operated by Analyst 1.7 HF3 acquisition software (SCIEX, Framingham MA). LC solvent is delivered by Prolab Zirconium micro-flow LC pump (Reinach, Switzerland). Mobile phase A consists of 0.1% formic acid in water, and mobile phase B consists of 0.1% formic acid in acetonitrile (Fisher Scientific). 30×0.3 mm micro-flow columns were obtained from Waters (Milford, MA).

Mobile phase was delivered to the system at a rate of 21 μL/min, with an initial composition of 97% A/3% B. Following sample injection, composition of mobile phase B increased to 85% over 30s, held at 85% for 5s, and immediately returned to initial conditions for column equilibration, lasting for 25s (total cycle time was 60s). Following sample analysis, multiply-injected LC-MS/MS chromatograms were integrated and processed in LeadScape (Review Analyze module).

Peak areas were corrected by dilution factors and incorporating internal standard, and the ratio of the corrected peak areas were used to calculate the results (log D value). The log D value for each compound was calculated using the following equation:

$$log {D}_{oct / buffer} = log ( \frac{{[ 200 - fold dilution of compound ]}_{octanol} \times 200}{{[ 20 - fold compound ]}_{buffer} \times 20} )$$

### Evaluation of Anti-Viral Activity Against RSV

### Plaque Reduction Assay:

The RSV plaque reduction assay is an infectivity assay which allows quantification of the number of infectious units in a distinct foci of RSV infection. As each plaque originates from a single infectious virus particle an accurate calculation of the anti-viral effect can be obtained by counting plaques in the presence and absence of an anti-viral compound. HepG2 cells (ECACC: 85011430) were passaged in flasks and seeded in 24-well plates in DMEM containing antibiotics and supplemented with 10% FBS. During inoculation and subsequent incubation, cells were cultured in DMEM containing 2% FBS. 100 plaque forming unit (PFU)/well of RSV (RSV A2 VR-1540) was mixed with serial dilutions of compound. Subsequently, 100 μL of the virus/compound mixtures was added to confluent HepG2 cell monolayers. Plates were incubated at 37° C. in a humidified 5% CO₂ incubator for 2 hrs. The inoculum was then removed and 1 ml of overlay (4% CMC in 2% DMEM) was added to each well. Cells were cultured for 48 h at 37° C. in a humidified 5% CO₂ incubator and then fixed with 75% acetone-25% methanol solution. Plates were washed under running water and blocking solution (2% skimmed milk powder in PBS-Tween) was added to each well and plates incubated on a shaker for 1 h at 37° C. Blocking solution was removed and 200 μl/well primary antibody (anti-RSV polyclonal antibody) in blocking solution was added and plates incubated for 90 min at 37° C. Plates were washed 2× under running water prior to addition of 200 ul/well secondary antibody (rabbit anti-goat HRP conjugate) in blocking solution and plates incubated for 1 h at 37° C. Plates were washed 2× under running prior to addition of 200 ul/well immunostaining reagent for 10 min at 37° C. Immunostaining reagent was then removed, plates washed 2× under running water and left to air dry, prior to being scanned on the CTL BioSpot® S6 Macro Analyser. Plaque counts were used to calculate % infection relative to the mean of the spot count in the virus control wells for RSV. The mean plaque number from duplicate wells of each compound dilution was plotted to construct a dose response curve and obtain EC₅₀ and EC₉₀ values. EC₉₀ values are provided in Table 2.

### In Vitro Determination of Clearance in Human Hepatocytes

Human hepatocyte clearance assay—Di, L., et al, Eur. J. Med. Chem. 2012, 57, 441-448.

The high throughput human hepatocyte stability assay was performed in a 384-well format. Pooled cryopreserved human hepatocytes of 10 donors were purchased from Celsis IVT (Baltimore, MD). The cryopreserved human hepatocytes were thawed, and re-suspended in Williams E medium (WEM GIBCO-BRL, cat #C1984, custom formula #91-5233EC) supplemented with HEPES and Na₂CO₃. The cells were counted using the Trypan Blue exclusion method. The Multidrop© liquid dispenser (Multidrop DW, Thermo Scientific, Waltham, MA) was used to add the hepatocyte suspensions to the 384-well plates. The cell plates were covered and transferred to a Sciclone® ALH 3000 workstation (Caliper Life Sciences, Hopkinton, MA), equipped with two 6-position Mecour heat exchangers. Test compounds were diluted on the Sciclone® with buffer and added to the hepatocytes. The final incubation contained 0.5 million cells/mL and 1 μM test compound in 15 μL total volume with 0.1% DMSO. The incubation was carried out at 37° C. At various time points (0, 3, 10, 30, 60, 120 min), reactions were quenched with cold acetonitrile containing internal standard (IS, CP-628374). The samples were centrifuged (Eppendorf, Hauppauge, NY) at 3000 rpm for 10 min at 4° C. The supernatants were transferred using the BioMek® liquid handler (Beckman Coulter, Inc. Danvers MA) to new plates, which were sealed prior to LC-MS/MS analysis. Detailed LC-MS/MS analysis conditions were described previously (Di, L., et al, J. Pharm. Sci. 2011, 100, 4974-4985). Propranolol (2D6, 1A2 and 2C₁₉ substrate), midazolam (CYP3A4) and triazolam (CYP3A4, low clearance) and naloxone (UGT2B7) were used as positive controls.

Relay Method Using Human Hepatocytes—Di, L., et al., Drug Metabolism and Disposition, 2012, 40, 1860-1865.

Pooled cryopreserved human hepatocytes of 10 donors were purchased from Celsis IVT (Baltimore, MD). This lot of pooled hepatocytes was used for all the studies. When selecting new lots of hepatocytes, enzyme activities need to be verified using marker compounds. Upon thawing, the hepatocytes were resuspended in Williams' medium E (custom formula number 91-5233EC; Invitrogen, Grand Island, NY) supplemented with HEPES and Na₂CO₃. The cells were counted using the trypan blue exclusion method, and the 24-well hepatocyte plates containing 0.5 million cells/ml were spiked with a compound at a final concentration of 1 μM (dimethyl sulfoxide, final concentration 0.025%; methanol, final concentration 0.125%), in a final incubation volume of 0.50 ml. The plates were covered with Breathe-Easy gas-permeable membranes (Diversified Biotech, Dedham, MA) and incubated at 37° C. with 95% O₂/5% CO₂, 75% relative humidity for 4 h at 150 rpm in a humidified incubator. At time 0 and 4 h, 25 μl of hepatocyte suspension was removed from the incubation and added to 50 μl of ice-cold acetonitrile containing internal standard to quench the reaction. The samples were centrifuged (Eppendorf, Hauppauge, NY) at 3000 rpm for 10 min at 4° C., and 50 μl of supernatant was transferred to a clean plate, dried completely, and reconstituted before liquid chromatography/tandem mass spectrometry (LC-MS/MS) analysis. The remaining hepatocyte suspensions in the incubation plate were centrifuged (3000 rpm, 10 min, 4° C.). The supernatant of 300 μl was transferred to a clean 24-well plate and stored at −80° C. until the next relay experiment. For the second relay experiment, the supernatant plates were warmed to 37° C. for 20 min, and hepatocytes were added to the samples to give a final cell density of 0.5 million cells/ml. The plates were incubated at 37° C. for 4 h, sampled, and processed as described above. Five relays were performed to give a total incubation time of 20 h. The supernatant from the last relay was saved in case more relays were needed for compounds with remarkably low clearance. Standard curves were prepared under the same conditions.

### LC-MS/MS Quantification.

The LC mobile phases were as follows: (A) HPLC grade water containing 0.1% formic acid, and (B) acetonitrile containing 0.1% formic acid. A solvent gradient from 5% (A) to 95% (B) over 2.0 min at the flow rate of 0.4 ml/min was used to elute the compounds from the column (Kinetex C18, 30×2 mm, 2.6 μm; Phenomenex, Torrance, CA). The cycle-time was 3 min/injection. A 5-μl aliquot of the sample was injected for analysis using a CTC PAL autosampler (LEAP Technology, Carrboro, NC). A full-scan mode from m/z 150 to 600 was applied to detect each compound. LCquan software (version 2.5; Thermo Fisher Scientific) was used for data collection, processing, and analysis. Terfenadine was used as an internal standard for LC-MS/MS quantification in positive ion multiple reaction monitoring mode. All of the test compounds had good linearity with R²>0.99, and the limit of quantitation was 1 nM for all of the compounds.

Human in vitro intrinsic clearance was calculated using methods as previously described in the reference above.

In vitro bone marrow assay—Chen, W., et al., J. Pharmacokinetics and Pharmacodynamics, 2020, 47, 163-182.

Primary human bone marrow mononuclear cells (Lonza) were cultured in stemline II hematopoietic stem cell expansion medium (Sigma Aldrich), supplemented with 5% FBS and under induction by the following cytokines (R&D Systems): 25 ng/mL stem cell factor (SCF), ng/mL G-CSF, 10 ng/mL granulocyte macrophage-colony stimulating factor, 3 U/mL erythropoietin (EPO), 15 ng/mL thrombopoietin (TPO), 10 ng/mL IL3, 10 ng/mL IL6, and ng/mL Flt3 ligand. Cell cultures were maintained in the 37° C., 5% CO₂, and 98% humidity incubator (see reference for further details). Cells were preincubated for 1 day and were then exposed to either DMSO or compound for 5 additional days. Cell counts were determined manually using a hemocytometer from 10 μL aliquots and converted to the total cell number by adjusting for the volume of the cell culture medium in each well. While the total cell count included myeloid, erythroid, and megakaryocytic lineages, neutrophil precursors comprised the majority of the total cell population given the stimulatory conditions of the cell culture. The effects on reduction of total cell counts, therefore, were used as a measure of anti-proliferation (see Hu W, Sung T, Jessen B A, Thibault S, Finkelstein M B, Khan N K, Sacaan A I (2016) Mechanistic investigation of bone marrow suppression associated with palbociclib and its differentiation from cytotoxic chemotherapies. Clin Cancer Res 22(8):2000-2008). Results are provided in Table 2.

### Comparative Anti-Viral Activity, Metabolic Stability, and Toxicity

Table 2 provides biological data for compound of Example 2 as well as Comparator compounds 1 to 3. Comparator compounds are the compounds of examples 25, 41, and 197 of WO2022008911 and can be prepared as described therein.

TABLE 2

| | structure | | | |
|---|---|---|---|---|
| | 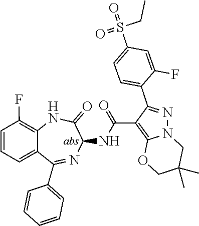 | 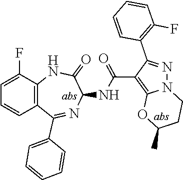 | 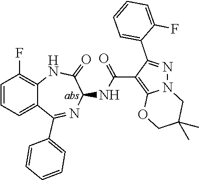 | 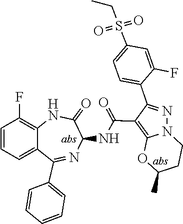 |
| com- | Example 2 | Comparator 1 | Comparator 2 | Comparator 3 |
| pound | of present | Ex. 25 | Ex. 41 | Ex. 197 |
| | invention | WO2022008911 | WO2022008911 | WO2022008911 |
| logD | 3.0 | 3.2 | 3.8 | 2.7 |
| RSV A2 | 3.5 | 25 | 27 | 5.7 |
| plaque, | | | | |
| HepG2, | | | | |
| EC₅₀ | | | | |
| (nM) | | | | |
| Human | <0.14* | 8.3 | 6.1 | 6.2 |
| Hepa- | | | | |
| tocyte | | | | |
| CL_{int,app} | | | | |
| (mL/min/ | | | | |
| million) | | | | |
| Human | >40000 | 3838 | 16722 | 42981 |
| Bone | | | | |
| Marrow | | | | |
| Tox | | | | |
| IC₅₀ | | | | |
| (nM) | | | | |

*Apparent intrinsic clearance (CL_{int,app}) value generated for Example 2 in human hepatocytes incubations conducted under relay conditions

High apparent intrinsic clearance (CL_{int,app}), measured in in vitro metabolic stability assays using liver microsomes and/or hepatocytes, is an undesirable trait of new chemical entities in a drug discovery program, manifesting as suboptimal pharmacokinetics and leading to unacceptable total dosing regimen(s) to elicit a pharmacologic effect. Optimization of metabolic clearance (CL_{int,app}) is an important consideration of potential clinical candidates, given its role in governing t_{1/2} and oral absorption. Modulation of lipophilicity, as represented by measurements of distribution coefficient (log D), remains a governing design principle in drug discovery. Higher lipophilicity positively correlates with CL_{int,app}, with values of log D<3 having greater probability of maintaining favorable metabolic stability as measured in a human hepatocyte (HHEP) assay. As can be seen in Table 2, removing the methyl group and substituting with a geminal di-methyl group, e.g., compare Comparator 1 to Comparator 2, increases log D, which results in similar moderate metabolic clearance values as assessed in in vitro HHEP assay measuring depletion of parent molecule with respect to time. Additionally, installation of an ethylsulfone moiety decreases log D by half a log unit, but the decrease in log D does not translate to a significant decrease in CL_{int,app}, (compare Comparator 1 with Comparator 3). When the combination of a 6,6-gem-dimethyl modification with installation of an ethylsulfone is made such as in the Example 2 of the present application, we observed a dramatic and unexpected improvement in metabolic stability studies in HHEP. Determination of the low CL_{int,app} value noted in Table 2 required evaluation in a HHEP assay under relay conditions to capture the slow metabolism process.

Installation of an ethylsulfone moiety also resulted in enhanced RSV EC₉o potency of compounds such as the Example 2 in this invention (compared to Comparator 1 or 2). The incorporation of ethylsulfone moiety and modification of the core possessing 6,6-gem-dimethyl motif led to concomitant improvements in toxicity as observed by the applicants when the compounds were evaluated by them in an in vitro human bone marrow assay. The in vitro bone marrow assay is used to evaluate a compound's effect on the proliferation of bone marrow progenitor cells with the effects on reduction of total cell counts being used as a measure of anti-proliferation. The in vitro results from the bone marrow toxicity assay have been shown to be predictive of neutropenia in reported clinical trials of Palbociclib in the reference above. In Table 2, Example 2 and Comparator 3 demonstrate favorable human bone marrow toxicity IC₅₀ in comparison to Comparator 1 and Comparator 2 suggesting improved safety for ethylsulfone analogs.

The specific combination of the geminal-methyl and ethylsulfone substitution provide for an unexpected simultaneous combination of improved properties (1) significantly enhanced potency against RSV N-protein, (2) significantly improved metabolic stability as assessed by human hepatocytes, and (3) improvement in safety as evaluated in a bone marrow toxicity assay. Example 2 is unique in this series of compounds in having this combined set of improvements relative to Comparators 1, 2, or 3. The impact of these changes results in an improved anti-viral agent with an unexpectedly low predicted human dose, improved safety and unexpectedly improved metabolic stability which is likely to translate in a longer pharmacokinetic half-life as compared to Comparators 1, 2, and 3.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. The compound of Formula (I) 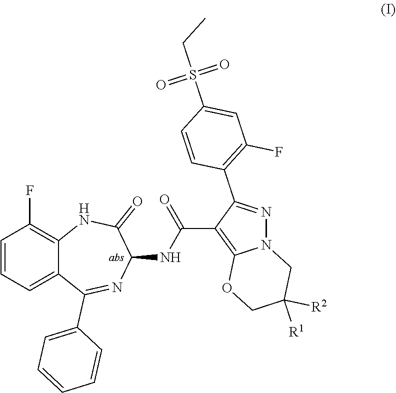
wherein R¹ or R² are independently selected from the group consisting of —CH₃, —CD₃, and —CH₂OH; or a pharmaceutically acceptable salt thereof.

2. The compound of claim 1 or a pharmaceutically acceptable salt thereof, wherein R¹ is —CH₃ or —CD₃, and R² is —CH₃, CD₃ or —CH₂OH.

3. The compound of claim 1 or a pharmaceutically acceptable salt thereof, wherein R¹ is —CH₃ and R² is —CH₃ or —CH₂OH.

4. The compound of claim 1 or a pharmaceutically acceptable salt thereof, wherein R¹ is —CH₃ and R² is —CH₂OH.

5. The compound of claim 4 that is a diastereomer represented by 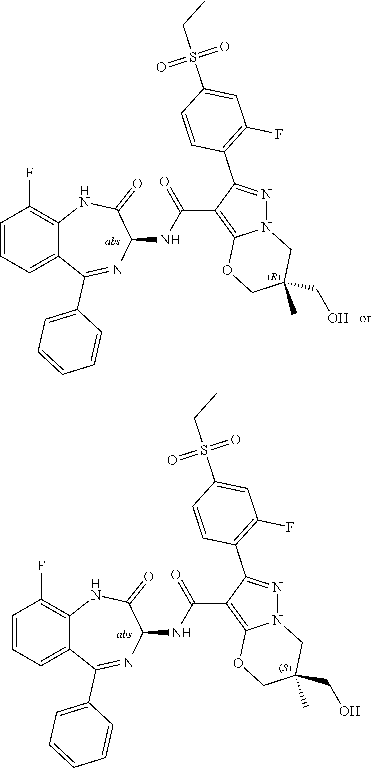
or pharmaceutically acceptable salt thereof.

6. The compound 2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide: 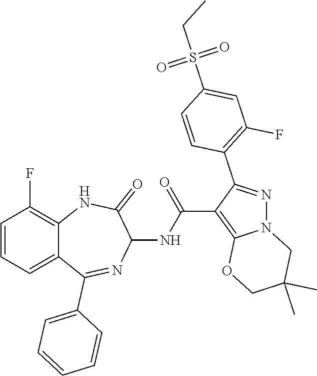 [SMILES: CCS(=O)(=O)c1ccc(-c2nn3c(c2C(=O)NC2N=C(c4ccccc4)c4cccc(F)c4NC2=O)OCC(C)(C)C3)c(F)c1]
or a pharmaceutically salt thereof.

7. The compound (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide: 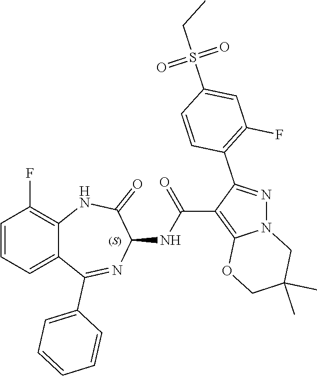
or a pharmaceutically acceptable salt thereof.

8. The compound that is (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide:

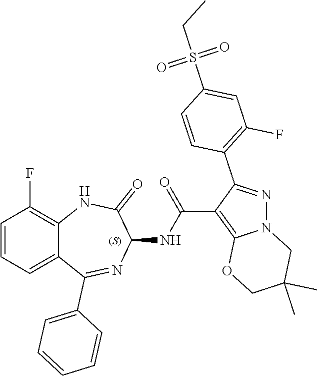

9. A pharmaceutically acceptable salt of (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide.

10. A pharmaceutical composition comprising the compound according to claim 1 or a pharmaceutically acceptable salt thereof, and one or more pharmaceutically acceptable excipients.

11. A pharmaceutical composition comprising a pharmaceutically acceptable salt of claim 1 and one or more pharmaceutically acceptable excipients.

12. A method for treating a RSV infection, the method comprising administering to a subject in need thereof a therapeutically effective amount of the compound or pharmaceutically acceptable salt thereof according to claim 1.

13. The method of claim 12 further comprising administering a therapeutically effective amount of an additional RSV therapeutic agent.

14. The method of claim 13 wherein the additional RSV therapeutic agent is selected from the group consisting of sisunatovir, ziresovir, EDP-938, EDP-323, JNJ-64417184, PC786, S-337395, MRK-1, JNJ-8003, BI-D, AVG-158, AVG-233, AZ-27, molnupiravir, remdesivir, obeldesivir, and ribavirin.

15. The method of claim 14 wherein the additional RSV therapeutic agent is selected from the group consisting of sisunatovir, ziresovir, EDP-938, EDP-323, JNJ-64417184, PC786, S-337395, MRK-1, JNJ-8003, BI-D, AVG-158, AVG-233, and AZ-27.

16. The method of claim 15 wherein the additional RSV therapeutic agent is sisunatovir.

17. The method of claim 12 wherein the compound is (S)-2-(4-(ethylsulfonyl)-2-fluorophenyl)-N-(9-fluoro-2-oxo-5-phenyl-2,3-dihydro-1H-benzo[e][1,4]diazepin-3-yl)-6,6-dimethyl-6,7-dihydro-5H-pyrazolo[5,1-b][1,3]oxazine-3-carboxamide.

18. The compound or pharmaceutically acceptable salt thereof according to claim 1 for use as a medicament.

19. The compound or pharmaceutically acceptable salt thereof according to claim 1 for use in the treatment of a RSV infection.

20. The compound or pharmaceutically acceptable salt thereof according to claim 1 for the manufacture of a medicament for the treatment of a RSV infection.
